(19) United States
(12) Patent Application Publication
(10) Pub. No.: US 2007/0118931 A1
(43) Pub. Date: May 24, 2007

(54) Plants having modified growth characteristics and method for making the same

(75) Inventors: Valerie Frankard, Sint-Genesius-Rode (BE); Vladimir Mironov, Gent (BE); Ana Isabel Sanz Molinero, Gentbrugge (BE)

Correspondence Address: DILWORTH & BARRESE, LLP, 333 EARLE OVINGTON BLVD., SUITE 702, UNIONDALE, NY, 11553, US

(73) Assignee: CROPDESIGN N.V., Zwijnaarde (BE)

(21) Appl. No.: 10/583,212

(22) PCT Filed: Dec. 17, 2004
(86) PCT No.: PCT/EP04/53594

§ 371 Date: Nov. 22, 2006

Related U.S. Application Data
(60) Provisional application No. 60/531,866, filed on Dec. 22, 2003.

(30) Foreign Application Priority Data
Dec. 17, 2003 (EP) 03104764.0

(51) Int. Cl. A01H 5/00; C12N 15/82

(52) U.S. Cl. 800/287; 800/320.2

(57) ABSTRACT

The present invention concerns a method for modifying the growth characteristics of plants by modulating expression in a plant of a nucleic acid sequence encoding a GRUBX protein and/or modulating activity and/or levels in a plant of a GRUBX protein. The present invention furthermore provides novel GRUBX proteins and nucleic acids encoding such proteins. The invention also relates to constructs comprising GRUBX encoding nucleic acids, and transgenic plants having modified growth characteristics, which plants have modulated expression of a nucleic acid encoding a GRUBX protein.

The present invention concerns a method for improving plant growth characteristics. More specifically, the present invention concerns a method for improving plant growth characteristics by modulating expression of a nucleic acid encoding a GRUBX protein and/or by modulating activity and/or levels of a GRUBX protein in a plant. The present invention furthermore provides novel GRUBX proteins and nucleic acids encoding such proteins. The present invention also concerns constructs comprising GRUBX encoding nucleic acids and plants having modulated expression of a nucleic acid encoding a GRUBX protein and/or modulated activity and/or levels of a GRUBX protein, which plants have improved growth characteristics relative to corresponding wild type plants.

Given the ever-increasing world population, and the dwindling area of land available for agriculture, it remains a major goal of agricultural research to improve the efficiency of agriculture and to increase the diversity of plants in horticulture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Furthermore, suitable donor species for providing a desired trait may be scarce. Advances in molecular biology have allowed mankind to manipulate the germplasm of animals and plants. Genetic engineering of plants entails the isolation and manipulation of genetic material (typically in the form of DNA or RNA) and the subsequent introduction of that genetic material into a plant. Such technology has led to the development of plants having various improved economic, agronomic or horticultural traits. Traits of particular economic interest are growth characteristics such as high yield. Yield is normally defined as the measurable produce of economic value from a crop. This may be defined in terms of quantity and/or quality. Crop yield is adversely influenced by the typical stresses to which plants or crops are subjected. Such stresses include abiotic stresses, such as temperature stresses caused by atypical high or low temperatures; stresses caused by nutrient deficiency; stresses caused by a lack of or excess water (drought, flooding), stresses caused by chemicals such as fertilisers or insecticides. Typical stresses also include biotic stresses, which may be imposed on plants by other plants (weeds, or the effects of high density planting), by animal pests (including stresses caused by grazing), and by pathogens. Crop yield may not only be increased by combating one or more of the stresses to which the crop or plant is subjected, but may also be increased by modifying the inherent growth mechanisms of a plant. The inherent growth mechanisms of a plant are controlled at several levels and by various metabolic processes. One such process is the control of protein levels in a cell by ubiquitin-mediated protein degradation.

Ubiquitination refers to a modification of proteins by conjugation to ubiquitin molecules. The term ubiquitination is often extended to processes that mediate binding of ubiquitin proteins or of proteins that mimic ubiquitin function. Ubiquitination is a versatile tool for eukaryotic cells to control stability, function and the subcelullar localisation of proteins. This mechanism plays a central role in protein degradation, cell cycle control, stress responses, DNA repair, signal transduction, transcriptional regulation and vesicular trafficking. Since ubiquitin mediated protein degradation is at the basis of many cellular processes, it is highly regulated and requires high substrate specificity and ample diversity in downstream effectors. Several ubiquitin-binding proteins are known. These proteins have often a modular domain architecture. For example, ubiquitin-binding proteins typically combine a ubiquitin binding domain with a variable effector domain. Then there are others that do not contain a ubiquitin binding domain, but have a tertiary structure similar to ubiquitin and can therefore mimic certain aspects of ubiquitination (ubiquitin-like domains).

The number of ubiquitin-related motifs and domains present in ubiquitin and ubiquitin-like proteins is growing as more information on genome sequences becomes available. Some prototypes of those domains are for example UBA, UBD, UIM and UBX (see for example the Pfam database; Bateman et al., Nucleic Acids Research 30(1):276-280 (2002)). The UBX domain is a sequence approximately 80 amino acid residues long, is of unknown function and is present in proteins of various organisms. Most of these proteins belong to one of five evolutionary conserved families exemplified by the human FAF1, p47, Y33K, REP8, and UBXD1 proteins (Buchberger et al. (2001) J. Mol. Biol. 307, 17-24; Carim-Todd et al. (2001) Biochim. Biophys. Acta 1517, 298-301). Typically, the UBX domain is situated at the C-terminus of a protein.

Structural evidence suggests a function of the UBX domain in ubiquitin-related processes; in particular the UBX domain may be involved in protein-protein interactions. Proteins comprising UBX domains are usually predicted to be present mainly in the cytoplasm, but other subcellular localizations have also been reported. For example, phosphorylation which is a specific protein modification used to regulate activity of many proteins, has been shown to also influence transport into the nucleus of FAF-1 (Olsen et al. (2003) FEBS Lett. 546, 218-222.). In summary, it has been proposed that animal UBX-containing proteins might be involved in

enhanced expression of genes related to apoptosis, cell cycling or targeting of proteins for degradation.

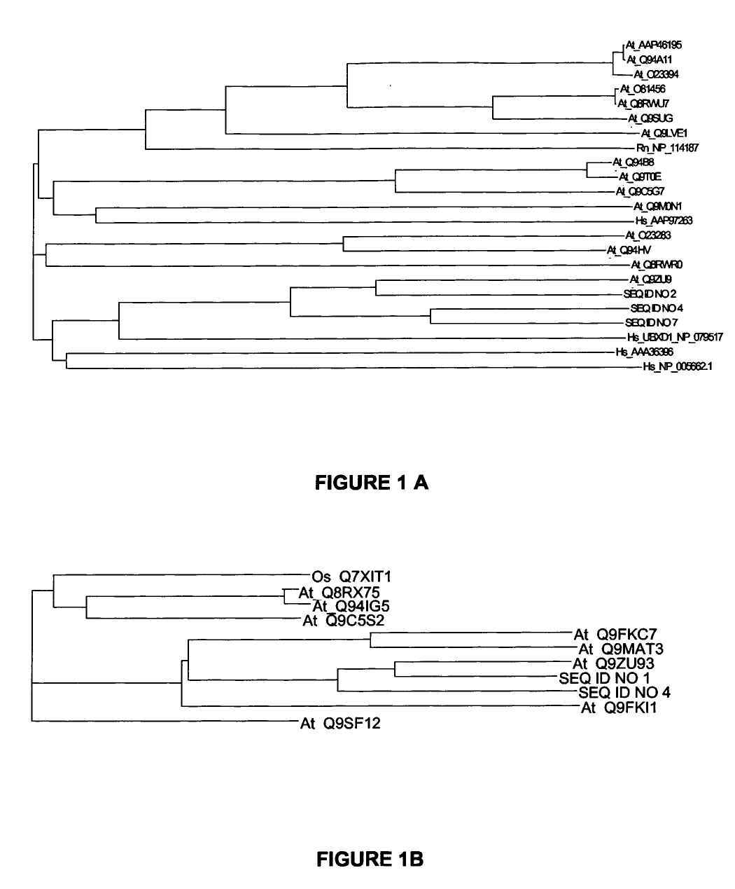

In Arabidopsis, the genome of which plant has been fully sequenced, there are at least 15 UBX-containing proteins. They may be classified according to sequence similarity in the FAF1, p47, Y33K and UBXF1 groups, only the group corresponding to REP8 appears not to be present in plants (see FIG. 1). As in the animal kingdom, the UBX domains in plant proteins are present in combination with other domains, like for example SEP, G6PD, PUG, or zinc fingers. UBX-containing proteins and the domain structure of these proteins have been described (see Buchberger (2002) Trends Cell Biol. 12, 216-221) and can be identified by searching using specialised databases such as SMART (Schultz et al. (1998) Proc. Natl. Acad. Sci. USA 95, 5857-5864; Letunic et al. (2002) Nucleic Acids Res 30, 242-244).

PUG domains (in Peptide:N-Glycanases and other putative nuclear UBX-domain-containing proteins; Doerks et al. (2002) Genome Research 12, 47-56) co-occur in proteins with domains that are central to ubiquitin-mediated proteolysis, including UBX (in mammals and plants), UBA (in plants) and UBC domains (in Plasmodium). PUG-containing proteins such as PNGases are believed to play a role in the unfolded protein response, an endoplasmatic reticulum (ER) quality control surveillance system that distinguishes aberrant proteins from correctly folded proteins. In some cases, it has been shown that these misfolded and/or unfolded proteins are degraded by a so-called ER-associated degradation mechanism, which involves the ubiquitin-proteasome system (Suzuki et al. (2000) J. Cell Biol. 149, 1039-1052). Divergent forms of PUG domains are also present in kinases of the IRE1p type which are known to function in the initial stages of the unfolded protein response (Shamu and Walter (1996) EMBO J. 15, 3028-3039).

A recently characterised Arabidopsis protein comprising an UBX domain is PUX1 (Rancour et al. (2004) J. Biol. Chem., online publication 10.1074/jbc.M405498200). PUX1 is a single gene in Arabidopsis and is probably expressed ubiquitously in planta. The protein was shown to be a non-competitive inhibitor of the AAA-type ATPase CDC48. PUX1 associates through its UBX domain with the non-hexameric form of CDC48, but not with the hexameric CDC48. It is postulated that PUX1 facilitates the disassembly of active hexameric CDC48 and that the N-terminal domain of the protein is required for this process. pux1 knockout plants showed a faster development to maturity but had no gross morphological abnormalities. Besides PUX1, two other UBX domain comprising proteins, PUX2 and PUX3, were shown to interact with CDC48 (Rancour et al., 2004). PUX2 (At2g01650) was previously disclosed in WO 03/085115 (gene and protein sequence described as SEQ ID NO: 1 and SEQ ID NO: 2 respectively).

It has now been found that modulating expression of a nucleic acid encoding a GRUBX protein (Growth Related UBX domain-comprising protein), and in particular a nucleic acid encoding the GRUBX protein exemplified by SEQ ID NO: 2, in a plant gives plants having improved growth characteristics. Therefore, according to a first embodiment of the present invention there is provided a method for improving the growth characteristics of a plant, comprising modulating expression in a plant of a nucleic acid encoding a GRUBX protein and/or modulating activity and/or levels in a plant of a GRUBX protein. According to a preferred aspect of the invention, the modulated expression is increased expression, the modulated activity and/or levels are increased activity and/or levels. Optionally, plants having improved growth characteristics may be selected for.

Modulating (enhancing or decreasing) expression of a nucleic acid encoding a GRUBX protein or modulation of the activity and/or levels of the GRUBX protein itself may result from altered expression of a gene and/or altered activity and/or levels of a gene product, namely a polypeptide, in specific cells or tissues. The modulated expression may result from altered expression levels of an endogenous GRUBX gene and/or may result from altered expression of a GRUBX encoding nucleic acid that was previously introduced into a plant. Similarly, modulated levels and/or activity of a GRUBX protein may be the result of altered expression levels of an endogenous GRUBX gene and/or may result from altered expression of a GRUBX encoding nucleic acid that was previously introduced into a plant. Activity may be increased when there is no change in levels of a GRUBX protein, or even when there is a reduction in levels of a GRUBX protein. This may be accomplished by altering the intrinsic properties, for example, by making a mutant that is more active than the wild type. Also encompassed is the inhibition or stimulation of regulatory sequences, or the provision of new regulatory sequences, that drive expression of the native gene encoding a GRUBX or the transgene encoding a GRUBX. Such regulatory sequences may be introduced into a plant. For example, the regulatory sequence introduced into the plant might be a promoter, capable of driving the expression of an endogenous GRUBX gene.

Expression of a gene, and activity and/or levels of a protein may be modulated by introducing a genetic modification (preferably in the locus of a GRUBX gene). The locus of a gene as defined herein is taken to mean a genomic region which includes the gene of interest and 10 kb up- or downstream of the coding region.

The genetic modification may be introduced, for example, by any one (or more) of the following methods: TDNA activation, TILLING, site-directed mutagenesis, homologous recombination or by introducing and expressing in a plant a nucleic acid encoding a GRUBX polypeptide or a homologue thereof. Following introduction of the genetic modification there follows a step of selecting for increased activity of a GRUBX polypeptide, which increase in activity gives plants having improved growth characteristics.

T-DNA activation tagging (Hayashi et aL Science (1992) 1350-1353) involves insertion of T-DNA usually containing a promoter (may also be a translation enhancer or an intron), in the genomic region of the gene of interest or 10 kB up- or downstream of the coding region of a gene in a configuration such that such promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to overexpression of genes near to the inserted T-DNA. The resulting transgenic plants show dominant phenotypes due to overexpression of genes close to the introduced promoter. The promoter to be introduced may be any promoter capable of directing expression of a gene in the desired organism, in this case a plant. For example, constitutive, tissue-specific, cell type-specific and inducible promoters are all suitable for use in T-DNA activation.

A genetic modification may also be introduced in the locus of a GRUBX gene using the technique of TILLING (Targeted Induced Local Lesions IN Genomes). This is a mutagenesis technology useful to generate and/or identify, and to eventually isolate mutagenised variants of a GRUBX nucleic acid capable of exhibiting GRUBX activity. TILLING also allows selection of plants carrying such mutant variants. These mutant variants may even exhibit higher GRUBX activity than that exhibited by the gene in its natural form. TILLING combines high-density mutagenesis with high-throughput screening methods. The steps typically followed in TILLING are: (a) EMS mutagenesis (Redei and Koncz (1992), In: C Koncz, N-H Chua, J Schell, eds, Methods in Arabidopsis Research. World Scientific, Singapore, pp 16-82; Feldmann et al., (1994) In: E M Meyerowitz, C R Somerville, eds, Arabidopsis. Cold Spring Harbor Laboratory Press, Cold Spring Harbor, N.Y., pp 137-172; Lightner and Caspar (1998), In: J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum, Nat Biotechnol. 2000 April; 18(4):455-7, Stemple, Nature Rev. Genet. 5, 145-150, 2004).

Site-directed mutagenesis may be used to generate variants of GRUBX nucleic acids or portions thereof that retain GRUBX activity, for example cation transporter activity. Several methods are available to achieve site-directed mutagenesis, the most common being PCR based methods (See for example Ausubel et al., Current Protocols in Molecular Biology. Wiley Eds. http://www.4ulr.com/products/currentprotocols/index.html).

TDNA activation, TILLING and site-directed mutagenesis are examples of technologies that enable the generation of novel alleles and variants of GRUBX that retain GRUBX function and which are therefore useful in the methods of the invention.

Homologous recombination allows introduction in a genome of a selected nucleic acid at a defined selected position. Homologous recombination is a standard technology used routinely in biological sciences for lower organism such as yeast and the moss Physcomitrella. Methods for performing homologous recombination in plants have been described not only for model plants (Offringa et al. (1990) EMBO J. 9, 3077-3084) but also for crop plants, for example rice (Terada et al., (2002) Nature Biotechnol. 20, 1030-1034; or lida and Terada (2004) Curr. Opin. Biotechnol. 15, 132-138). The nudeic acid to be targeted (which may be a GRUBX nucleic acid molecule or variant thereof as hereinbefore defined) need not be targeted to the locus of a GRUBX gene, but may be introduced in, for example, regions of high expression. The nucleic acid to be targeted may be an improved allele used to replace the endogenous gene or may be introduced in addition to the endogenous gene.

A preferred approach for modulating expression of a GRUBX gene, or modulating the activity and/or levels of a GRUBX protein, comprises introducing into a plant an isolated nucleic acid sequence encoding a GRUBX protein or a homologue, derivative or active fragment thereof. The nucleic acid may be introduced into a plant by, for example, transformation. Therefore, according to a preferred aspect of the present invention, there is provided a method for improving the growth characteristics of a plant comprising introducing and expressing a GRUBX encoding nucleic acid into a plant.

The term GRUBX protein, as defined herein, refers to a protein comprising at least an UBX domain, preferably an UBX and a PUG domain, and optionally also a Zinc finger domain. Preferably, the GRUBX protein is structurally related to the human UBXD1 protein (SPTrEMBL AAH07414). Preferably, the GRUBX protein is from a plant. Further preferably, the GRUBX protein is from the family of Solanaceae, more preferably the GRUBX is a protein from Nicotiana tabacum, most preferably the GRUBX is a protein as represented by SEQ ID NO: 2 or a homologue, derivative or active fragment thereof, which homologues, derivatives or active fragments have similar biological activity to that of SEQ ID NO: 2. However, it should be understood that GRUBX proteins from monocotyledonous plants could equally well be used in the methods of the present invention, including GRUBX proteins from Zea mays, Saccharum officinarum (SEQ ID NO 4), Oryza sativa (SEQ ID NO 7), Triticum sp., Hordeum sp., and Sorghum sp, since these sequences are related to SEQ ID NO 2 (see FIG. 1b).

One of the activities of a GRUBX protein is increasing seed yield, in particular increasing harvest index, when a nucleic acid encoding such GRUBX protein is expressed in rice under control of a prolamin promoter as used in the present invention. Advantageously, a GRUBX protein is able to interact with plant CDC48 proteins under conditions described in Rancour et al. (2004).

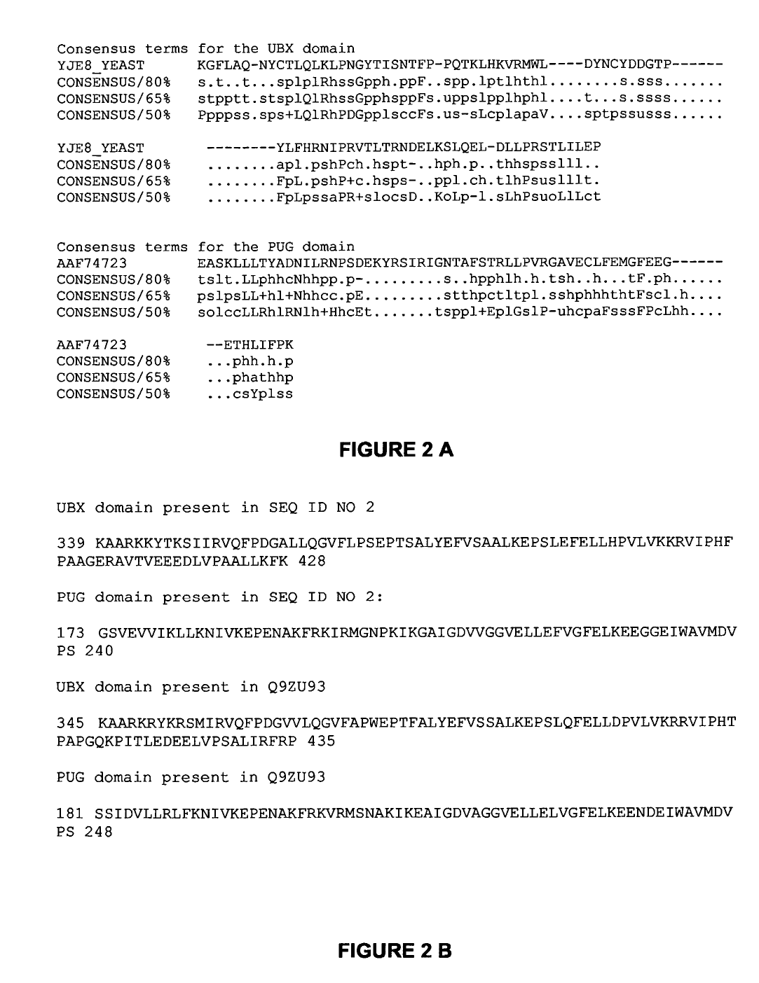

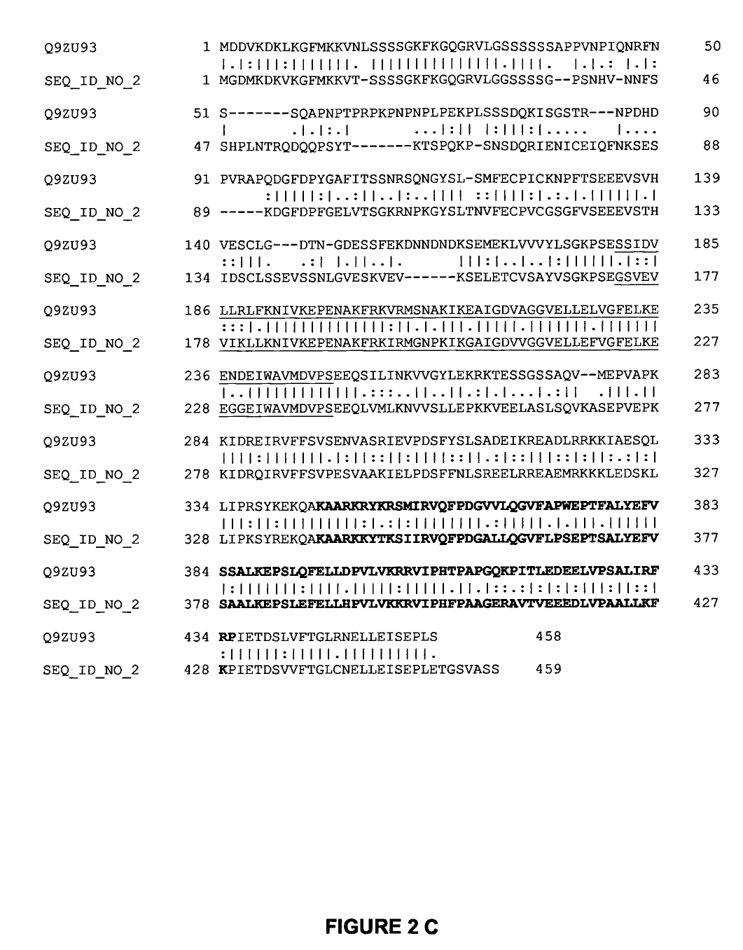

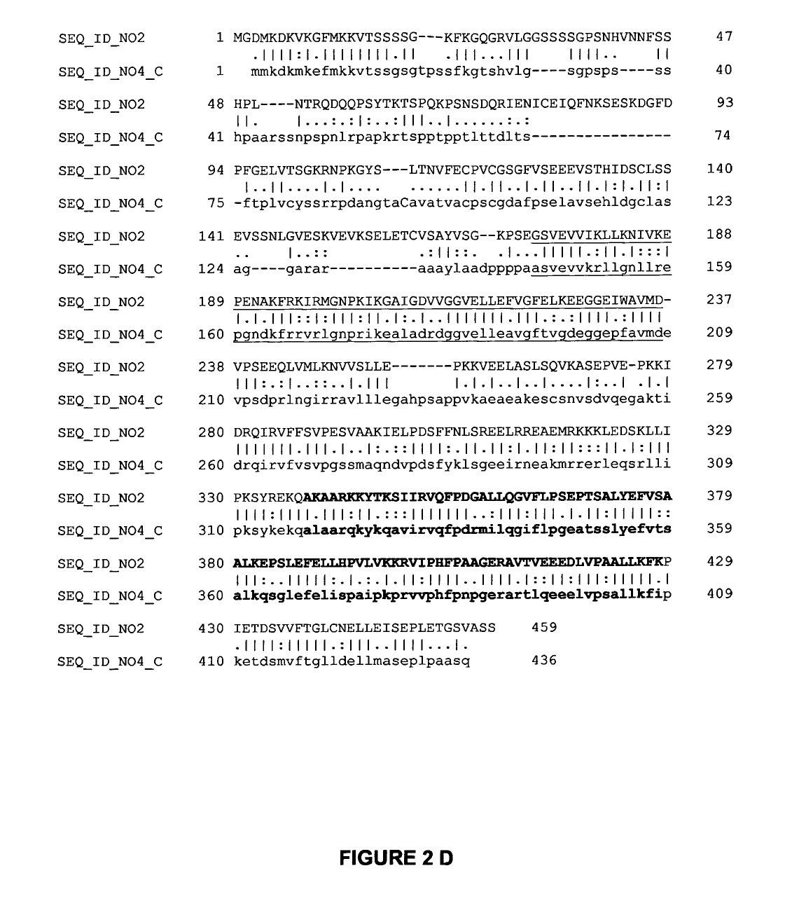

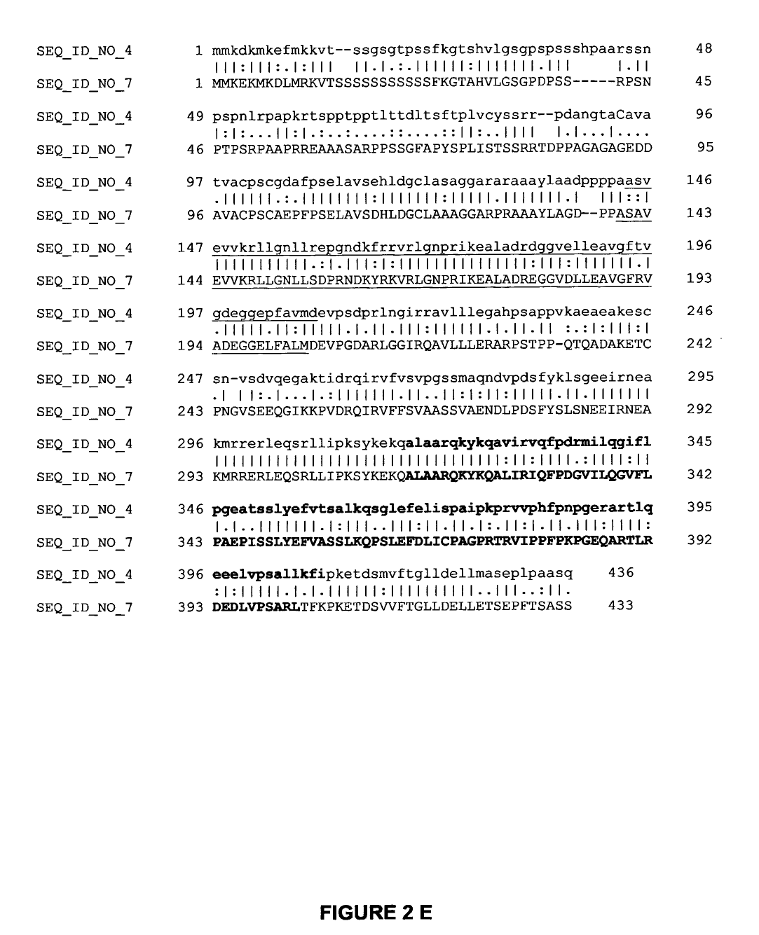

The GRUBX proteins of Nicotiana tabacum were analysed with the SMART tool and were used to screen the Pfam (Version 11.0, November 2003; Bateman et al. (2002) Nucl. Acids Res. 30, 276-280) and InterPro database (Release 7.0, 22 July 2003; Mulder et al. (2003) Nucl. Acids. Res. 31, 315-318). GRUBX proteins comprise an UBX domain (PF00789, SM00166, IPR001012) and a PUG domain (SM00580, IPR006567). The UBX domain, as defined in Interpro, is found in ubiquitin-regulatory proteins, which are members of the ubiquitination pathway, as well as a number of other proteins including FAF-1 (FAS-associated factor 1), the human Rep-8 reproduction protein and several hypothetical proteins from yeast. In Arabidopsis, there are approximately twenty proteins predicted to comprise this domain. The PUG domain is found in protein kinases, N-glycanases and other nuclear proteins in eukaryotes and is postulated to be involved in protein-protein interactions (for a review see Suzuki & Lennarz (2003) Biochem Biophys Res Commun. 302,1-5 and Biochem Biophys Res Commun. 303, 732) and in RNA binding (Doerks et al., 2002). PUG domains are often found together with UBA or UBX domains in Arabidopsis proteins (Doerks et al, 2002). A consensus sequence for the UBX and PUG domains, as defined in the SMART database (Software Version 4.0, sequence database update of 15 Sep. 2003) is given in FIG. 2a; FIG. 2b shows the UBX and PUG domains of respectively SEQ ID NO 2 and SPTrEMBL Q9ZU93; FIG. 2c shows a BLAST alignment of these 2 proteins; and FIGS. 2d and 2e display an alignment between SEQ ID NO 2 and SEQ ID NO 4, and SEQ ID NO 4 and SEQ ID NO 7, respectively. The PUG and UBX domains are indicated.

Optionally, a zinc finger domain may be present in the GRUBX protein. Zinc finger domains, as defined in InterPro, are nucleic acid-binding protein structures that were first identified in the Xenopus laevis transcription factor TFIIIA. These domains have since been found in numerous nucleic acid-binding proteins. A zinc finger domain is composed of 25 to 30 amino-acid residues including 2 conserved Cys and 2 conserved His residues in a C-2-C-12-H-3-H type motif. The 12 residues separating the second Cys and the first His are mainly polar and basic, indicating that this region is involved in nucleic acid binding. The zinc finger motif is an unusually small, self-folding domain in which Zn is a crucial component of its tertiary structure. All Zinc finger domains bind an atom of Zn in a tetrahedral array resulting in the formation of a finger-like projection which may interact with nucleotides in the major groove of the nucleic acid. The Zn binds to the conserved Cys and His residues. Fingers have been found to bind to about 5 base pairs of nucleic acid-containing short runs of guanine residues, and have the ability to bind to both RNA and DNA. The zinc finger may thus represent the original nucleic acid binding protein. It has also been suggested that a Zn-centred domain could be used in a protein interaction, for example in protein kinase C. Many classes of zinc fingers are characterized according to the number and positions of the histidine and cysteine residues involved in the spatial positioning of the zinc atom. In the first class to be characterized, called C2H2 (IPR007087), the first pair of zinc coordinating residues consists of cysteines, while the second pair are histidines. Another Zinc finger domain (IPR006642) may be of the type found in the Saccharomyces cerevisiae protein Rad18. Here too, the zinc finger domain is a putative nucleic acid binding sequence. The optional Zinc finger domain in the GRUBX protein as defined herein is however not restricted to the C2H2 or Rad18 type, but can be any type of Zinc finger domain.

The term GRUBX nucleic acid/gene, as defined herein, refers to any nucleic acid encoding a GRUBX protein, or the complement thereof. The nucleic acid may be derived (either directly or indirectly (if subsequently modified)) from any natural or artificial source provided that the nucleic acid, when expressed in a plant, leads to modulated expression of a GRUBX nucleic acid/gene or modulated activity and/or levels of a GRUBX protein. The nucleic acid may be isolated from a microbial source, such as bacteria, yeast or fungi, or from a plant, algal or animal (including human) source. Preferably the nucleic acid is derived from a eukaryotic organism. Preferably the GRUBX nucleic acid is of plant origin, further preferably of monocotyledonous or dicotyledonous plant origin, more preferably the GRUBX nucleic acid encodes a GRUBX protein from the family of Solanaceae, furthermore preferably the GRUBX nucleic acid is a nucleic acid sequence from Nicotiana tabacum, most preferably the GRUBX nucleic acid is a nucleic acid sequence as represented by SEQ ID NO: 1 or a functional portion thereof, or is a nucleic acid sequence capable of hybridising therewith, which hybridising sequence encodes a protein having GRUBX protein activity, i.e. similar biological activity to that of SEQ ID NO: 1, and also encompasses nucleic acids encoding an amino acid sequence represented by SEQ ID NO: 2 or homologues, derivatives or active fragments thereof. Alternatively, the nucleic acid encoding a GRUBX protein may be derived from the family of the Poaceae, preferably from Oryza saliva. This nucleic acid may be substantially modified from its native form in composition and/or genomic environment through deliberate human manipulation. The nucleic acid sequence is preferably a homologous nucleic acid sequence, i.e. a structurally and/or functionally related nudeic acid sequence, preferably obtained from a plant, whether from the same plant species or different.

The term “functional portion” refers to a portion of a GRUBX gene which encodes a polypeptide that retains the same biological activity of a GRUBX protein and that has an UBX domain, and preferably additionally a PUG domain, and optionally a Zinc finger domain. The term “GRUBX nucleic acid/gene” also encompasses a variant of the nucleic acid encoding a GRUBX protein due to the degeneracy of the genetic code, an allelic variant of the nucleic acid encoding a GRUBX, different splice variant of the nucleic acid encoding a GRUBX and variants that are interrupted by one or more intervening sequences.

Advantageously, the method according to the present invention may also be practised using portions of a nucleic acid sequence encoding a GRUBX protein (such as the sequence represented by SEQ ID NO: 1), or by using sequences that hybridise preferably under stringent conditions to a nucleic acid sequence encoding a GRUBX protein (which hybridising sequences encode proteins having GRUBX activity), or by using homologues, derivatives or active fragments of a GRUBX protein, such as the sequence according to SEQ ID NO: 2, or by using the nucleic acids encoding these homologues, derivatives or active fragments.

Homologues of GRUBX proteins such as the one represented in SEQ ID NO 2 may be found in various eukaryotic organisms. The closest homologues are generally found in the plant kingdom. The Arabidopsis thaliana genome seems to have at least 15 GRUBX proteins, of which the homologue with a sequence submitted in SPTrEMBL Q9ZU93 and Q8LGE5 (MIPS No. At2G01650, or GenBank AY084317 and AAM60904) is the closest homologue to SEQ ID NO: 2, other suitable homologues of SEQ ID NO: 2 include SEQ ID NO 4 from Saccharum officinarum, encoded by a nucleic acid represented in SEQ ID NO3, SEQ ID NO 7 (encoded by the nucleic acid sequence presented in SEQ ID NO 6) from Oryza sativa, and GenBank Accession Nos. BQ198347 and BF778922 from Pinus taeda.

Methods for the search and identification of GRUBX homologues would be well within the realm of persons skilled in the art. Such methods comprise comparison of the sequences represented by SEQ ID NO 1 or 2, in a computer readable format, with sequences that are available in public databases such as MIPS (Munich Information Center for Protein Sequences, http://mips.gsf.de/), GenBank (http://www.ncbi.nlm.nih.gov/Genbank/index.html) or EMBL Nudeotide Sequence Database (http://www.ebi.ac.uk/embl/index.html), using algorithms well known in the art for the alignment or comparison of sequences, such as GAP (Needleman and Wunsch, J. Mol. Biol. 48, 443-453 (1970)), BESTFIT (using the local homology algorithm of Smith and Waterman (Advances in Applied Mathematics 2, 482-489 (1981))), BLAST (Altschul, S. F., Gish, W., Miller, W., Myers, E. W. & Lipman, D. J., J. Mol. Biol. 215, 403-410 (1990)), FASTA and TFASTA (W. R. Pearson and D. J. Lipman Proc. Natl. Acad. Sci. USA 85, 2444-2448 (1988)). The software for performing BLAST analysis is publicly available through the National Centre for Biotechnology Information. The abovementioned homologues were identified using blast default parameters (for example BLASTN Program Advanced Options: G-Cost (to open a gap)=5; E-Cost (to extend a gap)=2; q-Penalty (for a mismatch)=−3; r-Reward (for a match)=1; e-Expectation value (E)=10.0; W-Word size=11; TBLASTN Program Advanced Options: G-Cost (to open a gap)=11; E-Cost (to extend a gap)=1; e-Expectation value (E)=10.0; W-Word size=3). As more genomes are being sequenced, it is expected that many more GRUBX homologues will be identifiable.

The sequence represented by SEQ ID NO: 6 was hitherto unknown. There is therefore provided in a second embodiment of the invention an isolated nucleic acid sequence comprising:
(a) a nucleic acid sequence represented by SEQ ID NO: 6, or the complement strand thereof;
(b) a nucleic acid sequence encoding an amino acid sequence represented by SEQ ID NO: 7, or homologues, derivatives or active fragments thereof;
(c) a nucleic acid sequence capable of hybridising (preferably under stringent conditions) with a nucleic acid sequence of (i) or (ii) above, which hybridising sequence preferably encodes a protein having GRUBX activity;
(d) a nucleic acid sequence according to (i) to (iii) above which is degenerate as a result of the genetic code;
(e) a nucleic acid which is an allelic variant of the nucleic acid sequences according to (a) to (d);
(f) a nucleic acid which is an alternative splice variant of the nudeic acid sequences according to (a) to (e);
(g) a nucleic acid sequence which has 75.00%, 80.00%, 85.00%, 90.00%, 95.00%, 96.00%, 97.00%, 98.00% or 99.00% sequence identity to any one or more of the sequence defined in (a) to (f);
(h) a portion of a nucleic acid sequence according to any one of (a) to (g) above, which portion preferably encodes a protein having GRUBX activity.

The sequence represented by SEQ ID NO: 4 was assembled from 4 EST sequences (CA154270, CA144028, BQ535511 & CA184742) and was hitherto unknown. There is therefore provided an isolated GRUBX protein selected from the group consisting of:
(i) a polypeptide as given in SEQ ID NO 4;
(ii) a polypeptide as given in SEQ ID NO 7;
(iii) a polypeptide with an amino acid sequence which has at least 40.00% sequence identity, preferably 50.00%, 60.00%, 70.00% sequence identity, more preferably 80% or 90% sequence identity, most preferably 95.00%, 96.00%, 97.00%, 98.00% or 99.00% sequence identity to the amino acid sequence as given in SEQ ID NO 4 or 7;
(iv) a polypeptide comprising at least an UBX domain, preferably an UBX and a PUG domain, and optionally a Zinc finger domain;
(v) a homologue, a derivative, an immunologically active and/or functional fragment of a protein as defined in any of (i) to (iv),
   with the proviso that the polypeptide sequence is not a sequence as represented by SEQ ID NO 2, or database entries Q9ZU93, AAR01744, Q9D7L9, Q9BZV1, Q99PL6, ENSANGP00000020442, Q7SXA8, Q9V8K8, Q96IK9, ENSRNOP00000037228, or AAH07414.

The term GRUBX includes proteins homologous to the GRUBX as presented in SEQ ID NO 2. Accordingly, preferred homologues to be used in the methods of the present invention comprise at least an UBX domain, preferably they comprise an UBX and a PUG domain. “Homologues” of a GRUBX protein encompass peptides, oligopeptides, polypeptides, proteins and enzymes having amino acid substitutions, deletions and/or insertions relative to the unmodified protein in question and having similar biological and functional activity as the unmodified protein from which they are derived. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W. H. Freeman and Company).

The homologues useful in the methods according to the invention have at least 40.00% sequence identity or similarity (functional identity) to the unmodified protein, alternatively at least 50.00% sequence identity or similarity to an unmodified protein, alternatively at least 60.00% sequence identity or similarity to an unmodified protein, alternatively at least 70.00% sequence identity or similarity to an unmodified protein. Typically, the homologues have at least 80% sequence identity or similarity to an unmodified protein, preferably at least 85.00% sequence identity or similarity, further preferably at least 90.00% sequence identity or similarity to an unmodified protein, most preferably at least 95.00%, 96.00%, 97.00%, 98.00% or 99.00% sequence identity or similarity to an unmodified protein. The percentage of identity can be calculated using alignment programs such as GAP. Despite what may appear to be a relatively low sequence homology (as low as approximately 40.00%), GRUBX proteins are highly conserved in structure, with all full-length proteins having at least an UBX domain, preferably an UBX domain and a PUG domain, and further optionally a Zinc finger domain. GRUBX proteins in other plant species may therefore easily be found (as evidenced by the above-mentioned novel sequences of rice and sugar cane).

Homologous proteins can be grouped in “protein families”. A protein family can be defined by functional and sequence similarity analysis, such as, for example, Clustal W. A neighbour-joining tree of the proteins homologous to GRUBX can be generated by the Clustal W program and gives a good overview of its structural and ancestral relationship (see for example FIGS. 1a and b, constructed with Vector NTI Suite 5.5, Informax). In a particular embodiment of the present invention, the GRUBX homologue(s) belong(s) to the same protein family as the protein corresponding to SEQ ID NO 2.

In the Arabidopsis genome a preferred family member of the GRUBX protein was identified (Q9ZU93, GenBank Refseq NM_{—}126226). Also in other plants such as rice, sugarcane or other monocotyledonous plants, family members of the GRUBX protein were identified as shown above. Advantageously also these family members are useful in the methods of the present invention.

Two special forms of homology, orthologous and paralogous, are evolutionary concepts used to describe ancestral relationships of genes. The term “paralogous” relates to homologous genes that result from one or more gene duplications within the genome of a species. The term “orthologous” relates to homologous genes in different organisms due to ancestral relationship of these genes. The term “homologues” as used herein also encompasses paralogues and orthologues of the proteins useful in the methods according to the invention. Orthologous genes can be identified by querying one or more gene databases with a query gene of interest, using for example the BLAST program. The highest-ranking subject genes that result from the search are then again subjected to a BLAST analysis, and only those subject genes that match again with the query gene are retained as true orthologous genes. If for example orthologues in rice were sought, the sequence in question would be blasted against the 28,469 full-length cDNA clones from Oryza sativa Nipponbare available at NCBI. BLASTn or tBLASTX may be used when starting from nucleotides or BLASTP or TBLASTN when starting from the protein, with standard default values. The blast results may be filtered. The full-length sequences of either the filtered results or the non-filtered results are then blasted back (second blast) against the sequences of the organism from which the sequence in question is derived, in casu Nicotiana tabacum. The results of the first and second blasts are then compared. An orthologue is found when the results of the second blast give as hits with the highest similarity a query GRUBX nucleic acid or GRUBX polypeptide. If for a specific query sequence the highest hit is found with a paralogue of GRUBX then such query sequence is also considered a homologue of GRUBX, provided that this homologue has GRUBX activity and comprises at least an UBX domain, preferably an UBX domain and a PUG domain, and optionally also a Zinc finger domain. The results may be further refined when the resulting sequences are analysed with ClustalW and visualised in a neighbour joining tree. The method can be used in identifying orthologues in many different species.

A further way to identify a functional orthologue within a group of related proteins is to determine the expression pattern and tissue distribution of the members of this protein family, whereby sequences present in the same tissues and with a similar expression pattern are expected to perform related functions. A further way to identify functional homologues of a protein is by identifying sequences with a similar conserved domain structure. Proteins carrying the same domains and particularly when the distribution of the domains is conserved, are expected to perform similar functions. Thus, similarities in chemical structure and in regulation (expression pattern, tissue specificity) could be useful to identify functional homologues of GRUBX.

“Homologues” of GRUBX encompass proteins having amino acid substitutions, insertions and/or deletions relative to the unmodified protein.

“Substitutional variants” of a protein are those in which at least one residue in an amino acid sequence has been removed and a different residue inserted in its place. Amino acid substitutions are typically of single residues, but may be clustered depending upon functional constraints placed upon the polypeptide; insertions will usually be of the order of about 1 to 10 amino acid residues, and deletions will range from about 1 to 20 residues. Preferably, amino acid substitutions comprise conservative amino acid substitutions.

“Insertional variants” of a protein are those in which one or more amino acid residues are introduced into a predetermined site in a protein. Insertions can comprise amino-terminal and/or carboxy-terminal fusions as well as intra-sequence insertions of single or multiple amino acids. Generally, insertions within the amino acid sequence will be smaller than amino- or carboxy-terminal fusions, of the order of about 1 to 10 residues. Examples of amino- or carboxy-terminal fusion proteins or peptides include the binding domain or activation domain of a transcriptional activator as used in the yeast two-hybrid system, phage coat proteins, (histidine)₆-tag, glutathione S-transferase-tag, protein A, maltose-binding protein, dihydrofolate reductase, Tag-100 epitope, c-myc epitope, FLAG®-epitope, lacZ, CMP (calmodulin-binding peptide), HA epitope, protein C epitope and VSV epitope.

“Deletion variants” of a protein are characterised by the removal of one or more amino acids from the protein. Amino acid variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

The term “derivatives” refers to peptides, oligopeptides, polypeptides, proteins and enzymes which may comprise substitutions, deletions or additions of naturally and non-naturally occurring amino acid residues compared to the amino acid sequence of a naturally-occurring form of the protein, for example, as presented in SEQ ID NO: 2 or 4. “Derivatives” of GRUBX encompass peptides, oligopeptides, polypeptides, proteins and enzymes which may comprise naturally occurring altered, glycosylated, acylated or non-naturally occurring amino acid residues compared to the amino acid sequence of a naturally-occurring form of the polypeptide. A derivative may also comprise one or more non-amino acid substituents compared to the amino acid sequence from which it is derived, for example a reporter molecule or other ligand, covalently or non-covalently bound to the amino acid sequence such as a reporter molecule which is bound to facilitate its detection, and non-naturally occurring amino acid residues relative to the amino acid sequence of a naturally-occurring protein.

“Active fragments” of a GRUBX protein encompasses at least 80 contiguous amino acid residues of a protein, which residues retain similar biological and/or functional activity to the naturally occurring protein. The active fragment at least comprises an UBX domain, preferably the active fragment comprise an UBX and a PUG domain.

Advantageously, the method according to the present invention may also be practised using portions of a DNA or nucleic acid sequence, which portions encode a polypeptide retaining GRUBX activity. Portions of a DNA sequence refer to a piece of DNA derived or prepared from an original (larger) DNA molecule, which DNA portion, when expressed in a plant, gives rise to plants having improved growth characteristics. The portion comprises at least 200 nucleotides, and comprises at least a sequence encoding an UBX domain, preferably an UBX domain and a PUG domain, and optionally a Zinc finger domain. A portion may be prepared, for example, by making one or more deletions to a GRUBX nucleic acid molecule. The portion may comprise many genes, with or without additional control elements, or may contain just spacer sequences etc. The portion may be in isolated form or it may be fused to other coding (or non-coding) sequences in order to, for example, produce a protein that combines several activities, one of them being increasing seed yield when expressed in plants under the control of a prolamin promoter. Preferably, the portion is of any one of SEQ ID NO: 1, SEQ ID NO: 3 or SEQ ID NO: 6.

The present invention also encompasses nucleic acid sequences capable of hybridising with a nucleic acid sequence encoding a GRUBX protein, which nucleic acid sequences may also be useful in practising the methods according to the invention. The term “hybridisation” as defined herein is a process wherein substantially homologous complementary nucleotide sequences anneal to each other. The hybridisation process can occur entirely in solution, i.e. both complementary nucleic acids are in solution. Tools in molecular biology relying on such a process indude the polymerase chain reaction (PCR; and all methods based thereon), subtractive hybridisation, random primer extension, nuclease S1 mapping, primer extension, reverse transcription, cDNA synthesis, differential display of RNAs, and DNA sequence determination. The hybridisation process can also occur with one of the complementary nucleic acids immobilised to a matrix such as magnetic beads, Sepharose beads or any other resin. Tools in molecular biology relying on such a process include the isolation of poly (A⁺) mRNA. The hybridisation process can furthermore occur with one of the complementary nucleic acids immobilised to a solid support such as a nitro-cellulose or nylon membrane or immobilised by, for example, photolithography to, for example, a siliceous glass support (the latter known as nucleic acid arrays or microarrays or as nucleic acid chips). Tools in molecular biology relying on such a process include RNA and DNA gel blot analysis, colony hybridisation, plaque hybridisation, in situ hybridisation and micro array hybridisation. In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration and hybridisation buffer composition.

For applications requiring high selectivity, one skilled in the art will typically desire to employ relatively stringent conditions to form the hybrids, for example, one will select relatively low salt and/or high temperature conditions, such as provided by about 0.02 M to about 0.15 M NaCl at temperatures of about 50° C. to about 70° C. High stringency conditions for hybridisation thus include high temperature and/or low salt concentration (salts include NaCl and Na₃-citrate) but may also be influenced by the inclusion of formamide in the hybridisation buffer and/or lowering the concentration of compounds such as SDS (sodium dodecyl sulphate) in the hybridisation buffer and/or exclusion of compounds such as dextran sulphate or polyethylene glycol (promoting molecular crowding) from the hybridisation buffer. Sufficiently low stringency hybridisation conditions are particularly preferred for the isolation of nucleic acids homologous to the DNA sequences of the invention defined supra. Elements contributing to homology include allelism, degeneration of the genetic code and differences in preferred codon usage.

“Stringent hybridisation conditions” and “stringent hybridisation wash conditions” in the context of nucleic acid hybridisation experiments such as Southern and Northern hybridisations are sequence dependent and are different under different environmental parameters. For example, longer sequences hybridise specifically at higher temperatures. The Tₘ is the temperature under defined ionic strength and pH, at which 50% of the target sequence hybridises to a perfectly matched probe. Specificity is typically the function of post-hybridisation washes. Critical factors of such washes indude the ionic strength and temperature of the final wash solution.

Generally, stringent conditions are selected to be about 50° C. lower than the thermal melting point (Tₘ) for the specific sequence at a defined ionic strength and pH. The Tₘ is the temperature under defined ionic strength and pH, at which 50% of the target sequence hybridises to a perfectly matched probe. The Tₘ is dependent upon the solution conditions and the base composition of the probe, and may be calculated using the following equation:
Tₘ=79.8° C.+(18.5×log[Na⁺])+(58.4° C.×%[G+C])−(820×(#bp in duplex)⁻)−(0.5×% formamide)

More preferred stringent conditions are when the temperature is 20° C. below Tₘ, and the most preferred stringent conditions are when the temperature is 10° C. below Tₘ. Non-specific binding may also be controlled using any one of a number of known techniques such as blocking the membrane with protein-containing solutions, additions of heterologous RNA, DNA, and SDS to the hybridisation buffer, and treatment with Rnase.

Wash conditions are typically performed at or below hybridisation stringency. Generally, suitable stringent conditions for nucleic acid hybridisation assays or gene amplification detection procedures are as set forth above. More or less stringent conditions may also be selected.

For the purposes of defining the level of stringency, reference can conveniently be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989). An example of low stringency conditions is 4-6×SSC/0.1-0.5% w/v SDS at 37-45° C. for 2-3 hours. Depending on the source and concentration of the nucleic acid involved in the hybridisation, alternative conditions of stringency may be employed such as medium stringent conditions. Examples of medium stringent conditions include 1-4×SSC/0.25% w/v SDS at ≧45° C. for 2-3 hours. An example of high stringency conditions includes 0.1-1×SSC/0.1% w/v SDS at 60° C. for 1-3 hours. The skilled artisan is aware of various parameters which may be altered during hybridisation and washing and which will either maintain or change the stringency conditions. For example, another stringent hybridisation condition is hybridisation at 4×SSC at 65° C., followed by a washing in 0.1×SSC, at 65° C. for about one hour. Alternatively, another stringent hybridisation condition is 50% formamide, 4×SSC, at 42° C. Still another example of stringent conditions include hybridisation at 62° C. in 6×SSC, 0.05× BLOTTO and washing at 2×SSC, 0.1% w/v SDS at 62° C.

The methods according to the present invention may also be practised using an alternative splice variant of a nucleic acid sequence encoding a GRUBX protein. The term “alternative splice variant” as used herein encompasses variants of a nucleic acid sequence in which selected introns and/or exons have been excised, replaced or added. Such variants will be ones in which the biological activity of the protein remains unaffected, which can be achieved by selectively retaining functional segments of the protein. Such splice variants may be found in nature or can be manmade. Methods for making such splice variants are well known in the art. Therefore according to another aspect of the present invention, there is provided, a method for improving the growth characteristics of plants, comprising modulating expression in a plant of an alternative splice variant of a nucleic acid sequence encoding a GRUBX protein and/or by modulating activity and/or levels of a GRUBX protein encoded by the alternative splice variant. Preferably, the splice variant is a splice variant of the sequence represented by SEQ ID NO: 1.

Advantageously, the methods according to the present invention may also be practised using allelic variants of a nucleic acid sequence encoding a GRUBX protein, preferably an allelic variant of a sequence represented by SEQ ID NO: 1. Allelic variants exist in nature and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp). SNPs and INDELs form the largest set of sequence variants in naturally occurring polymorphic strains of most organisms.

The use of these allelic variants in particular conventional breeding programmes, such as in marker-assisted breeding is also encompassed by the present invention; this may be in addition to their use in the methods according to the present invention. Such breeding programmes sometimes require the introduction of allelic variations in the plants by mutagenic treatment of a plant. One suitable mutagenic method is EMS mutagenesis. Identification of allelic variants then may take place by, for example, PCR. This is followed by a selection step for selection of superior allelic variants of the GRUBX sequence in question and which give rise to improved growth characteristics in a plant. Selection is typically carried out by monitoring growth performance of plants containing different allelic variants of the sequence in question, for example, different allelic variants of SEQ ID NO: 1. Monitoring growth performance can be done in a greenhouse or in the field. Further optional steps include crossing plants, in which the superior allelic variant was identified, with another plant. This could be used, for example, to make a combination of interesting phenotypic features. Therefore, as mutations in the GRUBX gene may occur naturally, they may form the basis for selection of plants showing higher yield. Accordingly, as another aspect of the invention, there is provided a method for the selection of plants having improved growth characteristics, which method is based on the selection of superior allelic variants of the GRUBX sequence and which give rise to improved growth characteristics in a plant.

The methods according to the present invention may also be practised by introducing into a plant at least a part of a (natural or artificial) chromosome (such as a Bacterial Artificial Chromosome (BAC)), which chromosome contains at least a gene/nudeic acid sequence encoding a GRUBX protein (such as SEQ ID NO: 1 or SEQ ID NO 3), preferably together with one or more related gene family members and/or nucleic acid sequence(s) encoding regulatory proteins for GRUBX expression and/or activity. Therefore, according to a further aspect of the present invention, there is provided a method for improving the growth characteristics of plants by introducing into a plant at least a part of a chromosome comprising at least a gene/nucleic acid encoding a GRUBX protein.

According to another aspect of the present invention, advantage may be taken of the nucleic acid encoding a GRUBX protein in breeding programmes. The nucleic acid sequence may be on a chromosome, or a part thereof, comprising at least the nucleic acid sequence encoding the GRUBX protein and preferably also one or more related family members. In an example of such a breeding programme, a DNA marker is identified which may be genetically linked to a gene capable of modulating expression of a nucleic acid encoding a GRUBX protein in a plant, which gene may be a gene encoding the GRUBX protein itself or any other gene which may directly or indirectly influence expression of the gene encoding a GRUBX protein and/or activity of the GRUBX protein itself. This DNA marker may then be used in breeding programs to select plants having improved growth characteristics.

The present invention therefore extends to the use of a nucleic acid sequence encoding a GRUBX protein in breeding programs.

GRUBX nucleic acids or variants thereof or GRUBX polypeptides or homologues thereof may find use in breeding programmes in which a DNA marker, a desired trait or a Quantitative Trait Locus (QTL), is identified which may be genetically linked to a GRUBX gene or variant thereof. This desirable trait or QTL may comprise a single gene or a cluster of linked genes that affect the desirable trait. The GRUBX or variants thereof or GRUBX or homologues thereof may be used to define a molecular marker. This DNA or protein marker may then be used in breeding programmes to select plants having improved growth characteristics. The GRUBX gene or variant thereof may, for example, be a nucleic acid as represented by SEQ ID NO: 1, or a nucleic acid encoding any of the above mentioned homologues.

Allelic variants of a GRUBX may also find use in marker-assisted breeding programmes. Such breeding programmes sometimes require introduction of allelic variation by mutagenic treatment of the plants, using for example EMS mutagenesis; alternatively, the programme may start with a collection of allelic variants of so-called “natural” origin (caused unintentionally). Identification of allelic variants then takes place by, for example, PCR. This is followed by a selection step for selection of superior allelic variants of the sequence in question and which give rise to improved growth characteristics in a plant, such as increased harvest index. Selection is typically carried out by monitoring growth performance of plants containing different allelic variants of the sequence in question, for example, different allelic variants of SEQ ID NO: 1, or of nucleic acids encoding any of the above mentioned plant homologues. Growth performance may be monitored in a greenhouse or in the field. Further optional steps include crossing plants, in which the superior allelic variant resulting in increased GRUBX activity was identified, with another plant. This could be used, for example, to make a combination of interesting phenotypic features.

A GRUBX nudeic acid or variant thereof may also be used as probes for genetically and physically mapping the genes that they are a part of, and as markers for traits linked to those genes. Such information may be useful in plant breeding in order to develop lines with desired phenotypes. Such use of GRUBX nucleic acids or variants thereof requires only a nucleic acid sequence of at least 10 nudeotides in length. The GRUBX nucleic acids or variants thereof may be used as restriction fragment length polymorphism (RFLP) markers. Southern blots of restriction-digested plant genomic DNA may be probed with the GRUBX nucleic acids, or variants thereof. The resulting banding patterns may then be subjected to genetic analyses using computer programs such as MapMaker (Lander et al. (1987) Genomics 1, 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the GRUBX nucleic acid or variant thereof in the genetic map previously obtained using this population (Botstein et al. (1980) Am. J. Hum. Genet. 32, 314-331).

The production and use of plant gene-derived probes for use in genetic mapping is described in Bematzky and Tanksley (Plant Mol. Biol. Reporter 4, 37-41, 1986). Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7, 149-154). Although current methods of FISH mapping favour use of large clones (several to several hundred kb; see Laan et al. (1995) Genome Res. 5, 13-20), improvements in sensitivity may allow performance of FISH mapping using shorter probes.

A variety of nucleic acid amplification-based methods of genetic and physical mapping may be carried out using the nucleic acids. Examples include allele-specific amplification (Kazazian (1989) J. Lab. Clin. Med. 11, 95-96), polymorphism of PCR-amplified fragments (CAPS; Sheffield et al. (1993) Genomics 16, 325-332), allele-specific ligation (Landegren et al. (1988) Science 241, 1077-1080), nucleotide extension reactions (Sokolov (1990) Nucleic Acid Res. 18, 3671), Radiation Hybrid Mapping (Walter et al. (1997) Nat. Genet. 7, 22-28) and Happy Mapping (Dear and Cook (1989) Nucleic Acid Res. 17, 6795-6807). For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

In this way, generation, identification and/or isolation of improved plants with altered GRUBX activity displaying improved growth characteristics can be performed.

According to another feature of the present invention, there is provided a method for improving plant growth characteristics, comprising modulating expression in a plant of a nucleic acid sequence encoding a GRUBX protein and/or modulating levels and/or activity of a GRUBX protein, wherein said nucleic acid sequence and said protein includes variants chosen from:
(i) an alternative splice variant of a nucleic acid sequence encoding a GRUBX protein or wherein said GRUBX protein is encoded by a splice variant;
(ii) an allelic variant of a nucleic acid sequence encoding a GRUBX protein or wherein said GRUBX protein is encoded by an allelic variant;
(iii) a nucleic acid sequence encoding a GRUBX protein and that is comprised on at least a part of an artificial chromosome, which artificial chromosome preferably also comprises one or more related gene family members;
(iv) a functional portion of a GRUBX encoding nucleic acid;
(v) sequence capable of hybridising to a GRUBX encoding nucleic acid;
(vi) homologues, derivatives and active fragments of a GRUBX protein.

According to a preferred aspect of the present invention, enhanced or increased expression of a nucleic acid is envisaged. Methods for obtaining enhanced or increased expression of genes or gene products are well documented in the art and include, for example, overexpression driven by a (strong) promoter, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a GRUBX nucleic acid or variant thereof. For example, endogenous promoters may be altered in vivo by mutation, deletion, and/or substitution (see Kmiec, U.S. Pat. No. 5,565,350; Zarling et al., PCT/US93/03868), or isolated promoters may be introduced into a plant cell in the proper orientation and distance from a gene of the present invention so as to control the expression of the gene. Preferably, the nucleic acids useful in the present invention are overexpressed in a plant or plant cell. The term overexpression as used herein means any form of expression that is additional to the original wild-type expression level. Preferably the nucleic acid to be introduced into the plant and/or the nucleic acid that is to be overexpressed in the plants is in a sense direction with respect to the promoter to which it is operably linked. Preferably, the nucleic acid to be overexpressed encodes a GRUBX protein, further preferably the nucleic acid sequence encoding the GRUBX protein is isolated from a dicotyledonous plant, preferably of the family Solanaceae, further preferably wherein the sequence is isolated from Nicotiana tabacum, most preferably the nucleic acid sequence is as represented by SEQ ID NO: 1 or a portion thereof, or encodes an amino acid sequence as represented by SEQ ID NO: 2 or a homologue, derivative or active fragment thereof. Alternatively, the nucleic acid sequence encoding the GRUBX protein is as represented by MIPS No. At2g01650, SEQ ID NO: 3 or 6, or is a portion thereof, or encodes an amino acid sequence as represented by Q9ZU93, SEQ ID NO: 4 or 7, or encodes a homologue, derivative or active fragment thereof. It should be noted that the applicability of the invention does not rest upon the use of the nucleic acid represented by SEQ ID NO: 1, nor upon the nucleic acid sequence encoding the amino acid sequence of SEQ ID NO: 2, but that other nucleic acid sequences encoding homologues, derivatives or active fragments of SEQ ID NO: 2, or portions of SEQ ID NO: 1, or sequences hybridising with SEQ ID NO: 1 may be used in the methods of the present invention. In particular, the nucleic acids useful in the methods of the present invention encode proteins comprising at least an UBX domain, preferably an UBX domain and a PUG domain, and optionally also a Zinc finger domain.

According to a further embodiment of the present invention, genetic constructs and vectors to facilitate introduction and/or expression of the nucleotide sequences useful in the methods according to the invention are provided. Therefore, according to a third embodiment of the present invention, there is provided a gene construct comprising:
(i) a nucleic acid encoding a GRUBX protein;
(ii) one or more control sequences capable of regulating expression of the nucleic acid sequence of (i); and optionally
(iii) a transcription termination sequence.
   provided that said nucleic acid encoding a GRUBX protein is not the nucleic acid represented in GenBank Accession number AX927140.

Constructs useful in the methods according to the present invention may be created using recombinant DNA technology well known to persons skilled in the art. The gene constructs may be inserted into vectors, which may be commercially available, suitable for transforming plants and suitable for expression of the gene of interest in the transformed cells. The genetic construct can be an expression vector wherein the nucleic acid sequence is operably linked to one or more control sequences allowing expression in prokaryotic and/or eukaryotic host cells.

According to a preferred embodiment of the invention, the genetic construct is an expression vector designed to overexpress the nucleic acid sequence. The nucleic acid sequence may be a nucleic acid sequence encoding a GRUBX protein or a homologue, derivative or active fragment thereof, such as any of the nucleic acid sequences described hereinbefore. A preferred nucleic acid sequence is the sequence represented by SEQ ID NO: 1 or a portion thereof or sequences capable of hybridising therewith or a nucleic acid sequence encoding a sequence represented by SEQ ID NO: 2 or a homologue, derivative or active fragment thereof. Preferably, this nucleic acid is cloned in the sense orientation relative to the control sequence to which it is operably linked.

Plants are transformed with a vector comprising the sequence of interest (i.e., the nucleic acid sequence capable of modulating expression of nucleic acid encoding a GRUBX protein), which sequence is operably linked to one or more control sequences (at least a promoter). The terms “regulatory element” , “control sequence” and “promoter” are all used herein interchangeably and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative which confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

Advantageously, any type of promoter may be used to drive expression of the nucleic acid sequence depending on the desired outcome. Suitable promoters include promoters that are active in monocotyledonous plants such as rice or maize.

Preferably, the nucleic acid sequence capable of modulating expression of a gene encoding a GRUBX protein is operably linked to a seed-preferred promoter. The term “seed-preferred” as defined herein refers to a promoter that is expressed predominantly in seed tissue, but not necessarily exclusively in this tissue. The term “seed-preferred” encompasses all promoters that are active in seeds. Seed tissue encompasses any part of the seed including the endosperm, aleurone or embryo. Preferably, the seed-preferred promoter is a prolamin promoter, or a promoter of similar strength and/or a promoter with a similar expression pattern. Most preferably, the prolamin promoter is as represented by nucleotides 1-654 in the expression cassette of SEQ ID NO: 5. Promoter strength and/or expression pattern can be analysed for example by coupling the promoter to a reporter gene and assay the expression of the reporter gene in various tissues of the plant. One suitable reporter gene well known to a person skilled in the art is bacterial beta-glucuronidase. Examples of other seed-preferred promoters are presented in Table 1, and these promoters are useful for the methods of the present invention.

TABLE 1
Examples of seed-preferred promoters for use in the
performance of the present invention:

| | EXPRESSION | |
|---|---|---|
| GENE SOURCE | PATTERN | REFERENCE |
| seed-specific genes | seed | Simon, et al., Plant Mol. Biol. 5: 191, |
| | | 1985; Scofield, et al., J. Biol. Chem. |
| | | 262: 12202, 1987.; Baszczynski, et al., |
| | | Plant Mol. Biol. 14: 633, 1990. |
| Brazil Nut albumin | seed | Pearson, et al., Plant Mol. Biol. 18: 235- |
| | | 245, 1992. |
| legumin | seed | Ellis, et al., Plant Mol. Biol. 10: 203-214, |
| | | 1988. |
| glutelin (rice) | seed | Takaiwa, et al., Mol. Gen. Genet. 208: |
| | | 15-22, 1986; Takaiwa, et al., FEBS |
| | | Letts. 221: 43-47, 1987. |
| zein | seed | Matzke et al Plant Mol Biol, 14(3): 323- |
| | | 32 1990 |
| napA | seed | Stalberg, et al, Planta 199: 515-519, |
| | | 1996. |
| wheat LMW and HMW | endosperm | Mol Gen Genet 216: 81-90, 1989; NAR |
| glutenin-1 | | 17: 461-2, 1989 |
| wheat SPA | seed | Albani et al, Plant Cell, 9: 171-184, 1997 |
| wheat α, β, γ-gliadins | endosperm | EMBO J. 3: 1409-15, 1984 |
| barley ltr1 promoter | endosperm | |
| barley B1, C, D, hordein | endosperm | Theor Appl Gen 98: 1253-62, 1999; |
| | | Plant J 4: 343-55, 1993; Mol Gen Genet |
| | | 250: 750-60, 1996 |
| barley DOF | endosperm | Mena et al, The Plant Journal, 116(1): |
| | | 53-62, 1998 |
| blz2 | endosperm | EP99106056.7 |
| synthetic promoter | endosperm | Vicente-Carbajosa et al., Plant J. 13: |
| | | 629-640, 1998. |
| rice prolamin NRP33 | endosperm | Wu et al, Plant Cell Physiology 39(8) |
| | | 885-889, 1998 |
| rice α-globulin Glb-1 | endosperm | Wu et al, Plant Cell Physiology 39(8) |
| | | 885-889, 1998 |
| rice OSH1 | embryo | Sato et al, Proc. Natl. Acad. Sci. USA, |
| | | 93: 8117-8122, 1996 |
| rice α-globulin REB/OHP-1 | endosperm | Nakase et al. Plant Mol. Biol. 33: 513- |
| | | 522, 1997 |
| rice ADP-glucose PP | endosperm | Trans Res 6: 157-68, 1997 |
| maize ESR gene family | endosperm | Plant J 12: 235-46, 1997 |
| sorgum γ-kafirin | endosperm | PMB 32: 1029-35, 1996 |
| KNOX | embryo | Postma-Haarsma et al, Plant Mol. Biol. |
| | | 39: 257-71, 1999 |
| rice oleosin | embryo and aleuron | Wu et at, J. Biochem., 123: 386, 1998 |
| sunflower oleosin | seed (embryo and dry | Cummins, et al., Plant Mol. Biol. 19: |
| | seed) | 873-876, 1992 |
| PRO0117, putative rice 40S | weak in endosperm | WO2004/070039 |
| ribosomal protein | | |
| PRO0135, rice alpha-globulin | strong in endosperm | |
| PRO0136, rice alanine | weak in endosperm | |
| aminotransferase | | |
| PRO0147, trypsin inhibitor | weak in endosperm | |
| ITR1 (barley) | | |
| PRO0151, rice WSI18 | embryo + stress | WO2004/070039 |
| PRO0175, rice RAB21 | embryo + stress | WO2004/070039 |
| PRO0218, rice oleosin 18 kd | aleurone + embryo | |

An intron sequence may also be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, Mol. Cell Biol. 8, 4395-4405 (1988); Callis et al., Genes Dev. 1, 1183-1200 (1987)). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences which may be suitable for use in performing the invention. Such sequences would be known or may readily be obtained by a person skilled in the art.

The genetic constructs of the invention may further include an origin of replication sequence which is required for maintenance and/or replication in a specific cell type. One example is when a genetic construct is required to be maintained in a bacterial cell as an episomal genetic element (for example plasmid or cosmid molecule). Preferred origins of replication include, but are not limited to, the f1-ori and colE1.

The genetic construct may optionally comprise a selectable marker gene. As used herein, the term “selectable marker gene” includes any gene which confers a phenotype on a cell in which it is expressed to facilitate the identification and/or selection of cells which are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA, allowing plants to use mannose as sole carbon source). Visual marker genes result in the formation of colour (for example β-glucuronidase, GUS), luminescence (such as luciferase) or fluorescence (Green Fluorescent Protein, GFP, and derivatives thereof).

In a preferred embodiment, the genetic construct as mentioned above, comprises a GRUBX in sense orientation coupled to a promoter that is preferably a seed-preferred promoter, such as for example the rice prolamin promoter. Therefore, another aspect of the present invention is a vector construct comprising an expression cassette essentially similar to SEQ ID NO 5, comprising a prolamin promoter, the Nicotiana tabacum GRUBX gene and the T-zein+T-rubisco deltaGA transcription terminator sequence. A sequence essentially similar to SEQ ID NO 5 encompasses a first nucleic acid sequence encoding a protein homologous to SEQ ID NO 2 or hybridising to SEQ ID NO 1, which first nucleic acid is operably linked to a prolamin promoter or a promoter with a similar expression pattern, additionally or alternatively the first nucleic acid is linked to a transcription termination sequence.

Therefore according to another aspect of the invention, there is provided a nucleic acid construct, comprising an expression cassette in which is located a nucleic acid sequence encoding a GRUBX protein, chosen from the group comprising:
(i) a nucleic acid sequence represented by SEQ ID NO: 1 or the complement strand thereof;
(ii) a nucleic acid sequence encoding an amino acid sequence represented by SEQ ID NO: 2 or homologues, derivatives or active fragments thereof;
(iii) a nucleic acid sequence capable of hybridising (preferably under stringent conditions) with a nucleic acid sequence of (i) or (ii) above, which hybridising sequence preferably encodes a protein having GRUBX protein activity;
(iv) a nucleic acid sequence according to (i) to (iii) above which is degenerate as a results of the genetic code;
(v) nucleic acid sequence which is an allelic variant of the nudeic acid sequences according to (i) to (iv);
(vi) nucleic acid sequence which is an alternative splice variant of the nucleic acid sequences according to (i) to (v).

The present invention also encompasses plants obtainable by the methods according to the present invention. The present invention therefore provides plants obtainable by the method according to the present invention, which plants have improved growth characteristics and which plants have altered GRUBX protein activity and/or levels and/or altered expression of a nucleic acid encoding a GRUBX protein, with the proviso that said GRUBX protein is not encoded by the nucleic acid sequence represented by the GenBank accession AX927140.

Thus, according to a fourth embodiment of the present invention, there is provided a method for the production of transgenic plants having improved growth characteristics, comprising introduction and expression in a plant of a nucleic acid molecule of the invention.

More specifically, the present invention provides a method for the production of transgenic plants having improved growth characteristics, which method comprises:
(a) introducing into a plant or plant cell a nucleic acid sequence, a nucleic acid sequence capable of hybridising therewith or a portion thereof, encoding a GRUBX protein or a homologue, derivative or active fragment thereof;
(b) cultivating the plant cell under conditions promoting plant growth.

The GRUBX protein itself and/or the GRUBX nucleic acid itself may be introduced directly into a plant cell or into the plant itself (including introduction into a tissue, organ or any other part of the plant). According to a preferred feature of the present invention, the nucleic acid is preferably introduced into a plant by transformation. The nucleic acid is preferably as represented by SEQ ID NO: 1 or a portion thereof or sequences capable of hybridising therewith, or is a nucleic acid encoding an amino acid sequence represented by SEQ ID NO: 2 or a homologue, derivative or active fragment thereof. Alternatively, the nucleic acid sequence is as represented by any of MIPS No. At2g01650, SEQ ID NO: 3, SEQ ID NO 6, or by a portion thereof or by sequences capable of hybridising with any of the aforementioned sequences. The amino acid sequence may alternatively be a sequence as represented by any of SPTrEMBL Q9ZU93, GenBank Acc. Nr. AAR01744, SEQ ID NO: 4, SEQ ID NO 7, or by homologues, derivatives or active fragments thereof.

The term “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (for example, apical meristem, axillary buds, and root meristems), and induced meristem tissue (for example, cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell can then be used to regenerate a transformed plant in a manner known to persons skilled in the art.

Transformation of a plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., 1882, Nature 296, 72-74; Negrutiu I. et al., June 1987, Plant Mol. Biol. 8, 363-373); electroporation of protoplasts (Shillito R. D. et al., 1985 Bio/Technol 3, 1099-1102); microinjection into plant material (Crossway A. et al., 1986, Mol. Gen Genet 202, 179-185); DNA or RNA-coated particle bombardment (Klein T. M. et al., 1987, Nature 327, 70) infection with (non-integrative) viruses and the like. Transgenic rice plants expressing a GRUBX gene are preferably produced via Agrobacterium-mediated transformation using any of the well known methods for rice transformation, such as described in any of the following: published European patent application EP 1198985 A1, Aldemita and Hodges (Planta, 199, 612-617, 1996); Chan et al. (Plant Mol. Biol. 22 (3) 491-506, 1993), Hiei et al. (Plant J. 6 (2) 271-282 1994), which disclosures are incorporated by reference herein as if fully set forth. In the case of corn transformation, the preferred method is as described in either Ishida et al. (Nat. Biotechnol. 1996 June; 14(6): 745-50) or Frame et al. (Plant Physiol. 2002 May; 129(1): 13-22), which disclosures are incorporated by reference herein as if fully set forth.

Generally after transformation, plant cells or cell groupings are selected for the presence of one or more markers which are encoded by plant-expressible genes co-transferred with the gene of interest, following which the transformed material is regenerated into a whole plant.

Following DNA transfer and regeneration, putatively transformed plants may be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed to give homozygous second-generation (or T2) transformants, and the T2 plants further propagated through classical breeding techniques.

The generated transformed organisms may take a variety of forms. For example, they may be chimeras of transformed cells and non-transformed cells; clonal transformants (for example, all cells transformed to contain the expression cassette); grafts of transformed and untransformed tissues (for example, in plants, a transformed rootstock grafted to an untransformed scion).

The present invention clearly extends to any plant cell or plant produced by any of the methods described herein, and to all plant parts, propagules and progeny thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. The invention also includes host cells containing an isolated nucleic acid molecule encoding a GRUBX protein. Preferred host cells according to the invention are plant cells. Therefore, the invention also encompasses host cells, transgenic plant cells or transgenic plants having improved growth characteristics, characterized in that said host cell, transgenic plant or plant cell has increased expression of a nucleic acid sequence encoding a GRUBX protein and/or increased activity and/or levels of a GRUBX protein.

The invention also extends to harvestable parts of a plant such as but not limited to seeds, leaves, fruits, flowers, stems or stem cultures, rhizomes, roots, tubers and bulbs, and to products directly derived thereof, such as dry pellets or powders, oil, fat and fatty acids, starch or proteins.

The term “plant” as used herein encompasses whole plants, ancestors and progeny of the plants, plant parts, plant cells, tissues and organs. The term “plant” also therefore encompasses suspension cultures, embryos, meristematic regions, callus tissue, leaves , flowers, fruits, seeds, roots (including rhizomes and tubers), shoots, bulbs, stems, gametophytes, sporophytes, pollen, and microspores. Plants that are particularly useful in the methods of the invention include algae, ferns, and all plants which belong to the superfamily Viridiplantae, in particular monocotyledonous and dicotyledonous plants, including fodder or forage legumes, ornamental plants, food crops, trees, or shrubs selected from the list comprising Abelmoschus spp., Acer spp., Actinidia spp., Agropyron spp., Allium spp., Amaranthus spp., Ananas comosus, Annona spp., Apium graveolens, Arabidopsis thaliana, Arachis spp, Artocarpus spp., Asparagus officinalis, Avena sativa, Averrhoa carambola, Benincasa hispida, Bertholletia excelsea, Beta vulgaris, Brassica spp., Cadaba farinosa, Camellia sinensis, Canna indica, Capsicum spp., Carica papaya, Carissa macrocarpa, Carthamus tinctorius, Carya spp., Castanea spp., Cichorium endivia, Cinnamomum spp., Citrullus lanatus, Citrus spp., Cocos spp., Coffee spp., Cola spp., Colocasia esculenta, Corylus spp., Crataegus spp., Cucumis spp., Cucurbita spp., Cynara spp., Daucus carota, Desmodium spp., Dimocarpus longan, Dioscorea spp., Diospyros spp., Echinochloa spp., Eleusine coracana, Eriobotrya japonica, Eugenia uniflora, Fagopyrum spp., Fagus spp., Ficus carica, Fortunella spp., Fragaria spp., Ginkgo biloba, Glycine spp., Gossypium hirsutum, Helianthus spp., Hibiscus spp., Hordeum spp., Ipomoea batatas, Juglans spp., Lactuca sativa, Lathyrus spp., Lemna spp., Lens culinaris, Linum usitatissimum, Litchi chinensis, Lotus spp., Luffa acutangula, Lupinus spp., Macrotyloma spp., Malpighia emarginata, Malus spp., Mammea americana, Mangifera indica, Manihot spp., Manilkara zapota, Medicago sativa, Melilotus spp., Mentha spp., Momordica spp., Morus nigra, Musa spp., Nicotiana spp., Olea spp., Opuntia spp., Omithopus spp., Oryza spp., Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Rubus spp., Saccharum spp., Sambucus spp., Secale cereale, Sesamum spp., Solanum spp., Sorghum bicolor, Spinacia spp., Syzygium spp., Tamarindus indica, Theobroma cacao, Trifollum spp., Triticosecale rimpaui, Triticum spp., Vaccinium spp., Vicia spp., Vigna spp., Vitis spp., Zea mays, Zizania palustris, Ziziphus spp., amongst others.

According to a preferred feature of the present invention, the plant is a crop plant comprising soybean, sunflower, canola, alfalfa, rapeseed or cotton. Further preferably, the plant according to the present invention is a monocotyledonous plant such as sugarcane, most preferably a cereal, such as rice, maize, wheat, millet, barley, rye, sorghum or oats. However, it is envisaged that the methods of the present invention can be applied to a wide variety of plants, since the domain conservation among the known eukaryotic GRUBX homologues suggests an equally conserved function in cellular metabolism.

Advantageously, performance of the methods according to the present invention results in plants having a variety of improved growth characteristics, such improved growth characteristics including improved growth, increased yield and/or increased biomass, modified architecture and a modified cell division, each relative to corresponding wild type plants.

The present invention relates to methods to improve growth characteristics of a plant or to methods to produce plants with improved growth characteristics, wherein the growth characteristics comprise any one or more selected from: increased yield, increased biomass, increased total above ground area, increased plant height, increased number of tillers, increased number of first panicles, increased number of second panicles, increased total number of seeds, increased number of filled seeds, increased total seed yield per plant, increased seed biomass, increased seed size, increased seed volume, increased harvest index, increased Thousand Kernel Weight (TKW), altered cycling time and/or an altered growth curve. The present invention also provides methods to alter one of the above mentioned growth characteristics, without causing a penalty on one of the other growth characteristics, for example increase of the above-ground green tissue area while retaining the same number of filled seeds and the same seed yield.

The term “increased yield” encompasses an increase in biomass in one or more parts of a plant relative to the biomass of corresponding wild-type plants. The term also encompasses an increase in seed yield, which includes an increase in the biomass of the seed (seed weight) and/or an increase in the number of (filled) seeds and/or in the size of the seeds and/or an increase in seed volume, each relative to corresponding wild-type plants. For maize, the increase of seed yield may be reflected in an increase of rows (of seeds) per ear and/or an increased number of kernels per row. Taking rice as an example, a yield increase may be manifested by an increase in one or more of the following: number of plants per hectare or acre, number of panicles per plant, number of spikelets per panicle, number of flowers per panicle, increase in the seed filling rate, among others. An increase in seed size and/or volume may also influence the composition of seeds. An increase in seed yield could be due to an increase in the number and/or size of flowers. An increase in yield might also increase the harvest index, which is expressed as a ratio of the total biomass over the yield of harvestable parts, such as seeds; or Thousand Kernel Weight. Increased yield also encompasses the capacity for planting at higher density (number of plants per hectare or acre).

The term “modified cell division” encompasses an increase or decrease in cell division or an abnormal cell division/cytokinesis, altered plane of division, altered cell polarity, altered cell differentiation. The term also comprises phenomena such as endomitosis, acytokinesis, polyploidy, polyteny and endoreduplication.

It can be envisaged that plants having increased biomass and height exhibit a modified growth rate when compared to corresponding wild-type plants. The term “modified growth rate” as used herein encompasses, but is not limited to, a faster rate of growth in one or more parts of a plant (including green biomass and including seeds), at one or more stages in the life cycle of a plant. The term “modified growth” encompasses enhanced vigour, earlier flowering, modified cycling time. If the growth rate is sufficiently increased, the resulting shorter cycling time may allow for an additional harvest within one conventional growing period. Harvesting additional times from the same root stock in the case of some plants may also be possible. Improving the harvest cycle of a plant may lead to an increase in annual biomass production per acre (due to an increase in the number of times (say in a year) that any particular plant may be grown and harvested. An increase in growth rate may also allow for the cultivation of modified plants in a wider geographical area than their wild-type counterparts, since the territorial limitations for growing a crop are often determined by adverse environmental conditions, either at the time of planting (early season) or at the time of harvesting (late season). Such adverse conditions may be avoided if the harvest cycle is shortened. Plants with modified growth may show a modified growth curve and may have modified values for their T_{mid} or T₉₀ (respectively the time needed to reach half of their maximal area or 90% of their area, each relative to corresponding wild-type plants).

According to a preferred feature of the present invention, performance of the methods according to the present invention result in plants having increased yield. Preferably, the increased yield indudes at least an increase in harvest index, relative to control plants. Therefore, according to the present invention, there is provided a method for increasing yield of plants, in particular harvest index, which method comprises increasing expression of a nucleic acid sequence encoding a GRUBX protein and/or increasing activity of a GRUBX protein itself in a plant, preferably wherein the GRUBX protein is encoded by a nucleic acid sequence represented by SEQ ID NO: 1 or a portion thereof or by sequences capable of hybridising therewith or wherein the GRUBX protein is represented by SEQ ID NO: 2 or a homologue, derivative or active fragment thereof. Alternatively, the GRUBX may be encoded by a nucleic acid sequence represented by any of MIPS No. At2g01650, SEQ ID NO: 3, or by a portion thereof or by sequences capable of hybridising therewith, or wherein the GRUBX is represented by any of SPTrEMBL Q9ZU93, SEQ ID NO: 4, or a homologue, derivative or active fragment of any thereof.

The methods of the present invention are favourable to apply to crop plants because the methods of the present invention are used to increase the harvest index of a plant. Therefore, the methods of the present invention are particularly useful for crop plants cultivated for their seeds, such as cereals. Accordingly, a particular embodiment of the present invention relates to a method to increase the harvest index of a cereal.

An increase in yield and/or growth occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Due to advances in agricultural practices (irrigation, fertilisation, pesticide treatments) severe stresses are not often encountered in cultivated crop plants. As a consequence, the compromised growth induced by mild stress is often an undesirable feature for agriculture. Mild stresses are the typical stresses to which a plant may be exposed. These stresses may be the everyday biotic and/or abiotic (environmental) stresses to which a plant is exposed. Typical abiotic or environmental stresses include temperature stresses caused by atypical hot or cold/freezing temperatures, salt stress, water stress (drought or excess water). Abiotic stresses may also be caused by chemicals. Biotic stresses are typically those stresses caused by pathogens, such as bacteria, viruses, fungi or insects.

“Modified architecture” may be due to change in cell division. The term “architecture” as used herein encompasses the appearance or morphology of a plant, including any one or more structural features or combination of structural features thereof. Such structural features include the shape, size, number, position, texture, arrangement, and pattern of any cell, tissue or organ or groups of cells, tissues or organs of a plant, including the root, leaf, shoot, stem or tiller, petiole, trichome, flower, inflorescence (for monocotyledonous and dicotyledonous plants), panicles, petal, stigma, style, stamen, pollen, ovule, seed, embryo, endosperm, seed coat, aleurone, fibre, cambium, wood, heartwood, parenchyma, aerenchyma, sieve elements, phloem or vascular tissue, amongst others. Modified architecture therefore includes all aspects of modified growth of the plant.

The present invention also relates to the use of a nucleic acid encoding a GRUBX protein and to the use of portions thereof or nucleic acids hybridising therewith in improving the growth characteristics of plants, preferably in increasing the yield and/or biomass of a plant. The present invention also relates to the use of a GRUBX protein and to the use of homologues, derivatives and active fragments thereof in improving the growth characteristics of plants. The nucleic acid sequence is preferably as represented by SEQ ID NO: 1, 6, or a portion thereof or sequences capable of hybridising therewith or encodes an amino acid sequence represented by SEQ ID NO: 2, 4, 7, or a homologue, derivative or active fragment thereof.

The present invention also relates to the use of a nucleic acid sequence encoding a GRUBX protein and variants thereof, and to the use of the GRUBX protein itself and of homologues, derivatives and active fragments thereof as growth regulators. The nucleic acid sequences hereinbefore described (and portions of the same and sequences capable of hybridising with the same) and the amino acid sequences hereinbefore described (and homologues, derivatives and active fragments of the same) are useful in improving the growth characteristics of plants, as hereinbefore described. The sequences would therefore find use as growth regulators, to stimulate or inhibit plant growth. Therefore, the present invention provides a composition comprising a GRUBX protein or a protein represented by SEQ ID NO 2 or a homologue, derivative or active fragment thereof for use in improving the growth characteristics of plants. The present invention furthermore provides a composition comprising a nucleic acid encoding a GRUBX protein, or a nucleic acid as represented by SEQ ID NO 1 or a portion thereof or a sequence hybridising therewith for use in improving the growth characteristics of plants. The present invention also provides a composition comprising a protein represented by any of the aforementioned amino acid sequences or homologues, derivatives or active fragments thereof for the use as a growth regulator.

The present invention will now be described with reference to the following figures in which:

FIG. 1a. Phylogenetic tree representing Arabidopsis thaliana proteins and animal reference proteins comprising an UBX domain, as recognised by the SMART tool. The human proteins are represented by their GenBank Accession numbers NP_{—}079517 (Homo sapiens UBX domain containing 1 (UBXD1)), AAP97263 (Homo sapiens Fas-associated protein factor FAF1 mRNA), NP_{—}005662 (Homo sapiens reproduction 8 (D8S2298E), REP8) and a rat protein by NP_{—}114187 (Rattus norvegicus p47 protein). The other identifiers (except for SEQ ID NO 2, SEQ ID NO 4 and SEQ ID NO 7) are GenBank or SPTrEMBL accession numbers for Arabidopsis thaliana proteins.

FIG. 1b. Phylogenetic tree representing plant proteins comprising a PUG domain, as recognised by the SMART tool. SEQ ID NO 2 and SEQ ID NO 4 are compared with Arabidopsis thaliana proteins (SPTrEMBL accessions Q9ZU93 (Expressed protein), Q9FKI1 (Similarity to zinc metalloproteinase), Q9MAT3 (F13M7.16 protein), Q9FKC7 (Genomic DNA, chromosome 5, TAC clone:K24G6), Q9SF12 (Hypothetical protein), Q9C5S2 (Endoribonuclease/protein kinase IRE1), Q8RX75 (AT5g24360/K16H17_{—}7), Q94IG5 (Ire1 homolog-1)), and with the rice protein SPTrEMBL Q7XIT1 (OsIre1p).

FIG. 2a. Definition of UBX1 and PUG domains by their consensus sequences (SMART database). CONSENSUS/50%, respectively /65% and /80% are the consensus sequences for the top 50, 65 and 80% of the reference sequences comprising the UBX1 or PUG domain.

The capital letters are the standard single letter IUPAC codes for the various amino acids, the other letters symbolise the nature of the amino acids as outlined below:

| Class | Key | Residues |
|---|---|---|
| Alcohol | o | S, T |
| Aliphatic | l | I, L, V |
| Any | . | A, C, D, E, F, G, H, I, K, L, M, N, P, |
| | | Q, R, S, T, V, W, Y |
| Aromatic | a | F, H, W, Y |
| Charged | c | D, E, H, K, R |
| Hydrophobic | h | A, C, F, G,H ,I, K, L, M, R, T, V, W, Y |
| Negative | − | D, E |
| Polar | p | C, D, E, H, K, N, Q, R, S, T |
| Positive | + | H, K, R |
| Small | s | A, C, D, G, N, P, S, T, V |
| Tiny | u | A, G, S |
| Turnlike | t | A, C, D, E, G, H, K, N, Q, R, S, T |

FIG. 2b. UBX and PUG domain sequences present in SEQ ID NO 2 and in Q9ZU93.

FIG. 2c. Alignment of Q9ZU93 and SEQ ID NO 2, PUG domains underlined, UBX domains in bold.

FIG. 2d. Alignment of SEQ ID NO 2 and SEQ ID NO 4, PUG domains underlined, UBX domains in bold.

FIG. 2e. Alignment of SEQ ID NO 4 and SEQ ID NO 7, PUG domains underlined, UBX domains in bold.

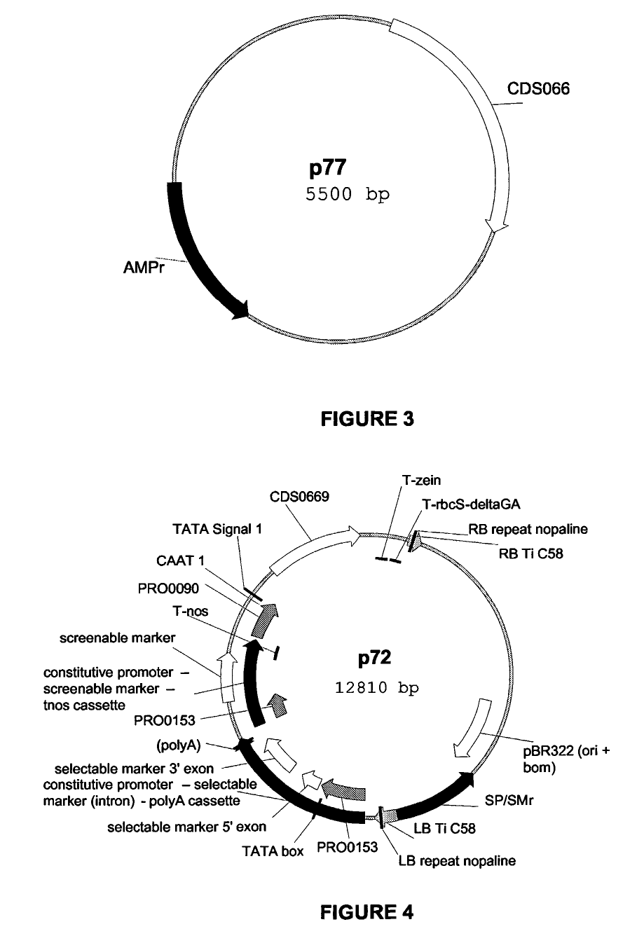

FIG. 3. Schematic presentation of the entry clone p77, containing CDS0669 within the AttL1 and AttL2 sites for Gateway® cloning in the pDONR201 backbone. CDS0669 is the internal code for the tobacco GRUBX coding sequence. This vector contains also a bacterial kanamycin-resistance cassette and a bacterial origin of replication.

FIG. 4. Binary vector for the expression in Oryza sativa of the tobacco GRUBX gene (CDS0669) under the control of the prolamin promoter (PRO0090). This vector contains a T-DNA derived from the Ti Plasmid, limited by a left border (LB repeat, LB Ti C58) and a right border (RB repeat, RB Ti C58)). From the left border to the right border, this T-DNA contains: a cassette for antibiotic selection of transformed plants; a cassette for visual screening of transformed plants; the PRO0090—CDS0669 -zein and rbcS-deltaGA double terminator cassette for expression of the tobacco GRUBX gene. This vector also contains an origin of replication from pBR322 for bacterial replication and a selectable marker (Spe/SmeR) for bacterial selection with spectinomycin and streptomycin.

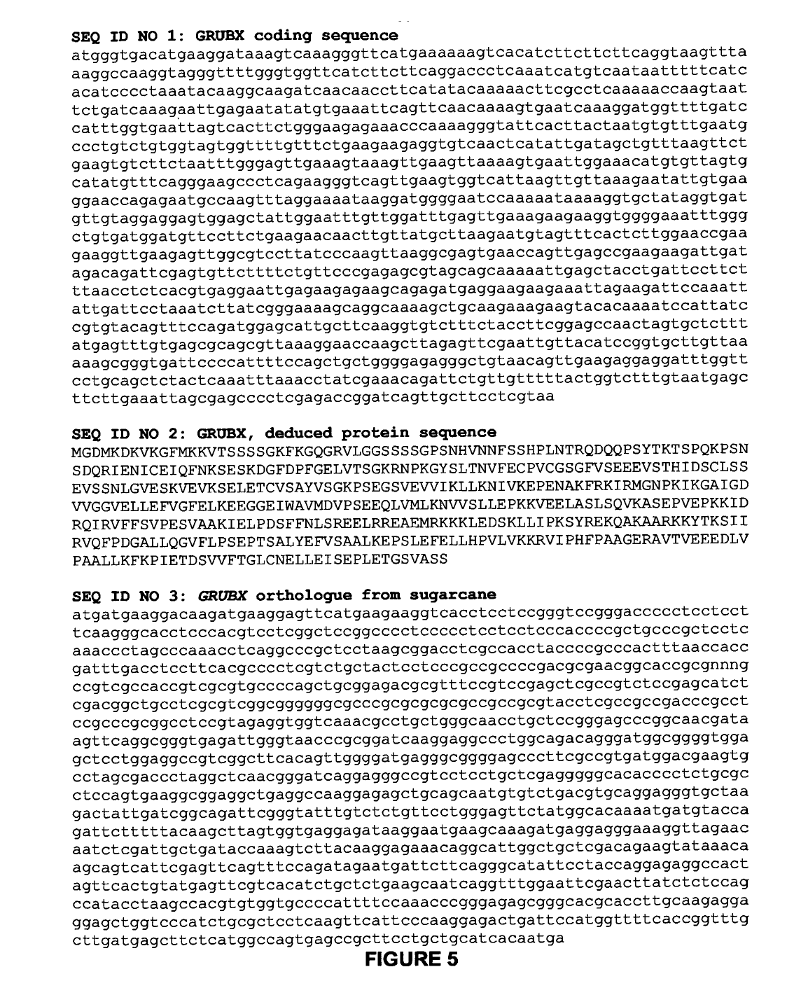

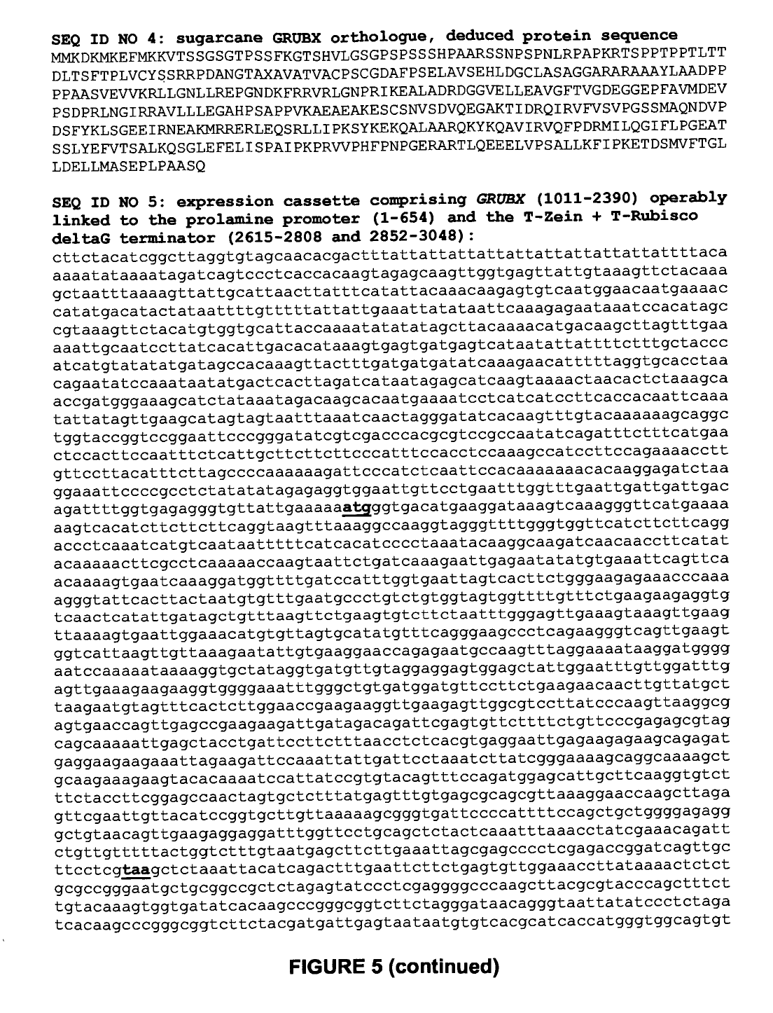

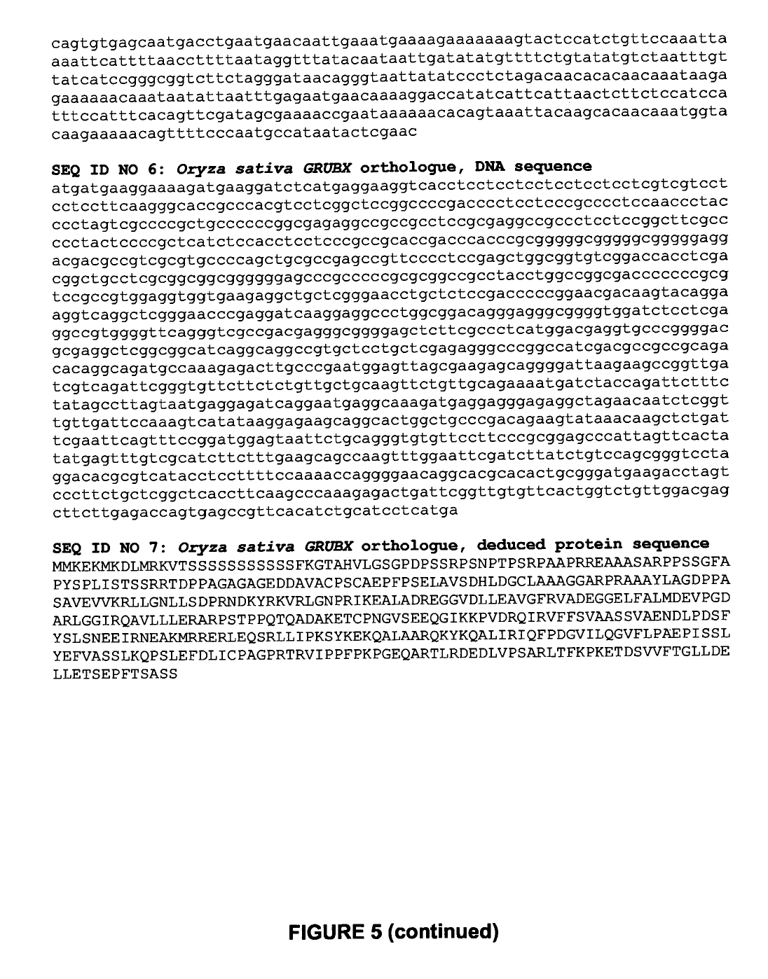

FIG. 5. Examples of sequences useful in the present invention. SEQ ID NO: 1 and SEQ ID NO: 2 are the sequences of the GRUBX nucleic acid and GRUBX protein respectively that were used in the examples. SEQ ID NO: 3 and SEQ ID NO: 4 represent the coding sequence and the protein sequence of the sugarcane GRUBX orthologue, SEQ ID NO: 5 is the sequence of the expression cassette that was used in the transformed rice plants, SEQ ID NO: 6 and SEQ ID NO: 7 represent the encoding sequence respectively protein sequence of the rice GRUBX orthologue.

### EXAMPLES

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (Current Protocols in Molecular Biology. New York: John Wiley and Sons, 1998). Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

Cloning of the CDS0669 Sequence

Cloning of the GRUBX Gene Fragment from Tobacco

A cDNA-AFLP experiment was performed on a synchronized tobacco BY2 cell culture (Nicotiana tabacum L. cv. Bright Yellow-2), and BY2 expressed sequence tags that were cell cycle modulated were elected for further cloning. The expressed sequence tags were used to screen a tobacco cDNA library and to isolate the full-length cDNA of interest, namely one coding for GRUBX gene (CDS0669).

Synchronization of BY2 Cells.

A tobacco BY2 (Nicotiana tabacum L. cv. Bright Yellow-2) cultured cell suspension was synchronized by blocking cells in early S-phase with aphidicolin as follows. The cell suspension of Nicotiana tabacum L. cv. Bright Yellow 2 was maintained as described (Nagata et al. Int. Rev. Cytol. 132, 1-30, 1992). For synchronization, a 7-day-old stationary culture was diluted 10-fold in fresh medium supplemented with aphidicolin (Sigma-Aldrich, St. Louis, Mo.; 5 mg/l), a DNA-polymerase α inhibiting drug. After 24 h, cells were released from the block by several washings with fresh medium after which their cell cycle progression resumed.

RNA Extraction and cDNA Synthesis.

Total RNA was prepared using LiCl precipitation (Sambrook et al, 2001) and poly(A⁺) RNA was extracted from 500 μg of total RNA using Oligotex columns (Qiagen, Hilden, Germany) according to the manufacturer's instructions. Starting from 1 μg of poly(A⁺) RNA, first-strand cDNA was synthesized by reverse transcription with a biotinylated oligo-dT₂₅ primer (Genset, Paris, France) and Superscript II (Life Technologies, Gaithersburg, Md.). Second-strand synthesis was done by strand displacement with Escherchia coli ligase (Life Technologies), DNA polymerase I (USB, Cleveland, Ohio) and RNAse-H (USB).

cDNA-AFLP Analysis.

Five hundred ng of double-stranded cDNA was used for AFLP analysis as described (Vos et al., Nucleic Acids Res. 23 (21) 44074-414, 1995; Bachem et al., Plant J. 9 (5) 745-53, 1996) with modifications. The restriction enzymes used were BstYI and Msel (Biolabs) and the digestion was done in two separate steps. After the first restriction digest with one of the enzymes, the 3′ end fragments were trapped on Dyna beads (Dynal, Oslo, Norway) by means of their biotinylated tail, while the other fragments were washed away. After digestion with the second enzyme, the released restriction fragments were collected and used as templates in the subsequent AFLP steps. For pre-amplifications, a Msel primer without selective nudeotides was combined with a BstYI primer containing either a T or a C as 3′ most nucleotide. PCR conditions were as described (Vos et al., 1995). The obtained amplification mixtures were diluted 600-fold and 5 μl was used for selective amplifications using a P³³-labeled BstYI primer and the Amplitaq-Gold polymerase (Roche Diagnostics, Brussels, Belgium). Amplification products were separated on 5% polyacrylamide gels using the Sequigel system (Biorad). Dried gels were exposed to Kodak Biomax films as well as scanned in a Phosphorlmager (Amersham Pharmacia Biotech, Little Chalfont, UK).

Characterization of AFLP Fragments.

Bands corresponding to differentially expressed transcripts, among which the (partial) transcript corresponding to SEQ ID NO 1 (or CDS0669), were isolated from the gel and eluted DNA was re-amplified under the same conditions as for selective amplification. Sequence information was obtained either by direct sequencing of the re-amplified polymerase chain reaction product with the selective BstYI primer or after cloning the fragments in pGEM-T easy (Promega, Madison, Wis.) and sequencing of individual clones. The obtained sequences were compared against nucleotide and protein sequences present in the publicly available databases by BLAST sequence alignments (Altschul et al., Nucleic Acids Res. 25 (17) 3389-3402 1997). When available, tag sequences were replaced with longer EST or isolated cDNA sequences to increase the chance of finding significant homology. The physical cDNA clone corresponding to SEQ ID NO 1 (CDS0669) was subsequently amplified from a commercial tobacco cDNA library as follows:

Cloning of the GRUBX Gene (CDS0669)

A c-DNA library with an average size of inserts of 1,400 bp was prepared from poly(A⁺) RNA isolated from actively dividing, non-synchronized BY2 tobacco cells. These library-inserts were cloned in the vector pCMVSPORT6.0, comprising an attB Gateway cassette (Life Technologies). From this library, 46,000 clones were selected, arrayed in 384-well microtiter plates, and subsequently spotted in duplicate on nylon filters. The arrayed clones were screened using pools of several hundreds of radioactively labelled tags as probes (including the BY2-tag corresponding to the sequence CDS0669, SEQ IDNO 1). Positive clones were isolated (among which the done corresponding to CDS0669, SEQ I NO 1), sequenced, and aligned with the tag sequence. Where the hybridisation with the tag failed, the full-length cDNA corresponding to the tag was selected by PCR amplification: tag-specific primers were designed using primer3 program (http://www-genome.wi.mit.edu/genome software/other/primer3.html) and used in combination with a common vector primer to amplify partial cDNA inserts. Pools of DNA from 50,000, 100,000, 150,000, and 300,000 cDNA clones were used as templates in the PCR amplifications. Amplification products were then isolated from agarose gels, cloned, sequenced and their sequence aligned with those of the tags. Next, the full-length cDNA corresponding to the nucleotide sequence of SEQ ID NO 1 was cloned from the pCMVsport6.0 library vector into pDONR201, a Gateway® donor vector (Invitrogen, Paisley, UK) via a LR reaction, resulting in the entry clone p77 (FIG. 3).

### Example 2

Vector Construction

The entry clone p77 was subsequently used in an LR reaction with p0830, a destination vector used for Oryza sativa transformation. This vector contained as functional elements within the T-DNA borders: a plant selectable marker; a visual marker expression cassette; and a Gateway cassette intended for LR in vivo recombination with the sequence of interest already cloned in the entry clone. A prolamin promoter for seed-preferred expression (PRO0090) was upstream of this Gateway cassette. After the LR recombination step, the resulting expression vector p72 (FIG. 4) was transformed into Agrobacterium strain LBA4404 and subsequently into Oryza sativa plants.

### Example 3

Transformation of Rice with the PRO0090-CDS0669 Construct

Mature dry seeds of Oryza sativa japonica cultivar Nipponbare were dehusked. Sterilization was done by incubating the seeds for one minute in 70% ethanol, followed by 30 minutes in 0.2% HgCl₂ and by 6 washes of 15 minutes with sterile distilled water. The sterile seeds were then germinated on a medium containing 2,4-D (callus induction medium). After a 4-week incubation in the dark, embryogenic, scutellum-derived calli were excised and propagated on the same medium. Two weeks later, the calli were multiplied or propagated by subculture on the same medium for another 2 weeks. 3 days before co-cultivation, embryogenic callus pieces were sub-cultured on fresh medium to boost cell division activity. The Agrobacterium strain LBA4414 harbouring binary vector p72 was used for co-cultivation. The Agrobacterium strain was cultured for 3 days at 28° C. on AB medium with the appropriate antibiotics. The bacteria were then collected and suspended in liquid co-cultivation medium at an OD₆₀₀ of about 1. The suspension was transferred to a petri dish and the calli were immersed in the suspension for 15 minutes. Next, the callus tissues were blotted dry on a filter paper, transferred to solidified co-cultivation medium and incubated for 3 days in the dark at 25° C. Thereafter, co-cultivated callus was grown on 2,4-D-containing medium for 4 weeks in the dark at 28° C. in the presence of a selective agent at a suitable concentration. During this period, rapidly growing resistant callus islands developed. Upon transfer of this material to a regeneration medium and incubation in the light, the embryogenic potential was released and shoots developed in the next four to five weeks. Shoots were excised from the callus and incubated for 2 to 3 weeks on an auxin-containing medium from which they were transferred to soil. Hardened shoots were grown under high humidity and short days in a greenhouse. Finally seeds were harvested three to five months after transplanting. The method yielded single locus transformants at a rate of over 50% (Aldemita and Hodges, Planta 199, 612-617, 1996; Chan et al., Plant Mol. Biol. 22(3), 491-506, 1993; Hiei et al., Plant J. 6(2), 271-282, 1994).

### Example 4

Evaluation of Transgenic Rice Transformed with the PRO0090-CDS0669 Construct

Approximately 15 to 20 independent T0 rice transformants were generated. The primary transformants were transferred from tissue culture chambers to a greenhouse for growing and harvest of T1 seed. 6 events, of which the T1 progeny segregated 3:1 for presence/absence of the transgene, were retained. For each of these events, approximately 10 T1 seedlings containing the transgene (hetero- and homo-zygotes), and approximately 10 T1 seedlings lacking the transgene (nullizygotes), were selected by monitoring visual marker expression. A number of parameters related to vegetative growth and seed production were evaluated and all data were statistically analysed as outlined below:

Statistical Analysis: T-test and F-test:

A two factor ANOVA (analysis of variants) was used as statistical model for the overall evaluation of plant phenotypic characteristics. An F-test was carried out on all the parameters measured of all the plants of all the events transformed with the gene of the present invention. The F-test is carried out to check for an effect of the gene over all the transformation events and to verify for an overall effect of the gene, also named herein “global gene effect”. If the value of the F-test shows that the data are significant, than it is concluded that there is a “gene” effect, meaning that not only presence or the position of the gene is causing the differences in phenotype. The threshold for significance for a true global gene effect is set at 5% probability level for the F-test.

4.1 Vegetative Growth Measurements:

The selected T1 plants (approximately 10 with the transgene and approximately 10 without the transgene) were transferred to a greenhouse. Each plant received a unique barcode label to link unambiguously the phenotyping data to the corresponding plant. The selected T1 plants were grown on soil in 10 cm diameter pots under the following environmental settings: photoperiod=11.5 h, daylight intensity=30,000 lux or more, daytime temperature=28° C. or higher, night time temperature=22° C., relative humidity=60-70%. Transgenic plants and the corresponding nullizygotes were grown side-by-side at random positions. From the stage of sowing until the stage of maturity each plant was passed several times through a digital imaging cabinet and imaged. At each time point digital images (2048×1536 pixels, 16 million colours) were taken of each plant from at least 6 different angles. Several parameters can be derived in an automated way from all the digital images of all the plants, using image analysis software.

4.2 Seed-Related Parameter Measurements:

The mature primary panicles were harvested, bagged, barcode-labelled and then dried for three days in the oven at 37° C. The panicles were then threshed and all the seeds were collected and counted. The filled husks were separated from the empty ones using an air-blowing device. The empty husks were discarded and the remaining fraction was counted again. The filled husks were weighed on an analytical balance. This procedure allows to derive a set of seed-related parameters.

Harvest Index of Plants

The harvest index in the present invention is defined as the ratio between the total seed yield and the above ground area (mm²), multiplied by a factor 10⁶. The total seed yield per plant was measured by weighing all filled husks harvested from a plant as described above. Plant aboveground area was determined by counting the total number of pixels of the digital images from aboveground plant parts discriminated from the background. This value was averaged for the pictures taken on the same time point from the different angles and was converted to a physical surface value expressed in square mm by calibration. Experiments showed that the aboveground plant area measured this way correlates with the biomass of plant parts above ground.

The data obtained in the first experiment were confirmed in a second experiment with T2 plants. Three lines that had the correct expression pattern were selected for further analysis. Seed batches from the positive plants (both hetero- and homozygotes) in T1, were screened by monitoring marker expression. For each chosen event, the heterozygote seed batches were then retained for T2 evaluation. Within each seed batch an equal number of positive and negative plants were grown in the greenhouse for evaluation.

A total number of 120 GRUBX transformed plants were evaluated in the T2 generation, that is 40 plants per event of which 20 positives for the transgene, and 20 negatives.

Because two experiments with overlapping events have been carried out, a combined analysis was performed. This is useful to check consistency of the effects over the two experiments, and if this is the case, to accumulate evidence from both experiments in order to increase confidence in the conclusion. The method used was a mixed-model approach that takes into account the multilevel structure of the data (i.e. experiment—event—segregants). P-values are obtained by comparing likelihood ratio test to chi square distributions.

In a first experiment, six lines in T1 generation were evaluated. There was an average increase of the harvest index and two lines had a significant increase of 50% or more compared to the nullizygote lines (Table 2).

TABLE 2
Evaluation of the two best performing T1 events
Harvest index:

| Line | TR | null | dif | % dif | p-value |
|---|---|---|---|---|---|
| 10 | 74.9 | 49.9 | 24.97 | 50 | 0.039 |
| 4 | 35 | 21.7 | 13.28 | 61 | 0.0656 |

Mean absolute values of the measurements of harvest index for the transgenic lines (TR) and control plants (null) in the T1 generation are given in columns 2 and 3, the absolute difference in column 4 and the difference in % in column 5, significance, expressed as a p-value obtained in a t-test, is given in column 6.

The results obtained for the T1 generation were confirmed in the T2 generation; the average increase for harvest index was 13% and an F-test showed this increase was significant (p-value of 0.0447). Furthermore, these T2 data were re-evaluated in a combined analysis with the results for the T1 generation, and the p-value obtained from an F-test showed again that the observed effects were significant (p-value 0.0181).

1. A method for improving plant growth characteristics, said method comprising increasing expression in a plant of a nucleic acid sequence encoding a GRUBX protein and/or comprising increasing activity and/or increasing levels in a plant of a GRUBX protein, and optionally selecting for plants having improved growth characteristics.

2. The method of claim 1, wherein said increase is effected by introducing a genetic modification, preferably in the locus of a gene encoding a GRUBX protein.

3. The method according to claim 2, wherein said genetic modification is effected by one of site-directed mutagenesis, homologous recombination, TILLING and T-DNA activation.

4. A method for improving plant growth characteristics, said method comprising introducing and expressing in a plant an isolated nucleic acid molecule encoding a GRUBX protein.

5. The method according to claim 4, wherein said nucleic acid molecule encoding a GRUBX protein is overexpressed in a plant.

6. The method according to claim 4, wherein said nucleic acid molecule is derived from a eukaryotic organism, preferably from a plant.

7. The method according to claim 6, wherein said nucleic acid molecule is derived from a dicotyledonous plant, preferably from the family Solanaceae, more preferably from Nicotiana tabacum.

8. The method according to claim 6, wherein said nucleic acid is molecule derived from a monocotyledonous plant, preferably from the family Poaceae, more preferably from Oryza sativa.

9. The method according to claim 7, wherein said nucleic acid molecule is as represented by SEQ ID NO: 1 or is a portion thereof or is a sequence capable of hybridising therewith or encodes a GRUBX protein, wherein said GRUBX protein is represented by SEQ ID NO: 2 or a homologue, derivative or active fragment thereof.

10. The method according to any of claims 4 to 9, wherein said nucleic acid molecule and said proteins include variants chosen from:
(i) an alternative splice variant of a nucleic acid sequence encoding a GRUBX protein or wherein said GRUBX protein is encoded by a splice variant;
(ii) an allelic variant of a nucleic acid sequence encoding a GRUBX protein or wherein said GRUBX protein is encoded by an allelic variant;
(iii) a nucleic acid sequence that is comprised on at least a part of an artificial chromosome, which artificial chromosome preferably also comprises one or more related gene family members;
(iv) a functional portion of a GRUBX encoding nucleic acid;
(v) sequence capable of hybridising to a GRUBX encoding nucleic acid;
(vi) homologues, derivatives and active fragments of a GRUBX protein.

11. The method according claim 10, wherein expression of said nucleic acid molecule encoding a GRUBX protein is driven by a seed-preferred promoter, preferably a prolamin promoter.

12. The method according to claim 11, wherein said improved growth characteristic is increased yield and/or modified plant architecture, each relative to corresponding wild type plants.

13. The method according to claim 12, wherein said increased yield is increased seed yield.

14. The method according to claim 13, wherein said increased yield and said modified plant architecture comprise one or more of (i) increased seed biomass, (ii) increased total number of seeds, (iii) increased number of filled seeds, (iv) increased seed size, (v) increased seed volume, (vi) increased harvest index, and (vii) increased Thousand Kernel Weight, all relative to corresponding wild type plants.

15. A method for increasing the yield of a plant, which method comprises increasing expression in a plant of a GRUBX encoding nucleic acid and/or increasing activity and/or levels in a plant of a GRUBX protein.

16. A method for the production of a transgenic plant having improved growth characteristics, which method comprises:
a. introducing into a plant or plant cell a nucleic acid sequence, a nucleic acid sequence capable of hybridising therewith or a portion thereof, encoding a GRUBX protein or a homologue, derivative or active fragment thereof;
b. cultivating the plant cell under conditions promoting plant growth.

17. A method for the selection of plants having improved growth characteristics, which method is based on the selection of superior allelic variants of a GRUBX encoding sequence and which alleles give rise to improved growth characteristics in a plant.

18. Plants obtained by a method according to any of claims 1 to 9, with the proviso that said GRUBX protein is not encoded by the nucleic acid sequence represented by the GenBank accession AX927140.

19. An isolated nucleic acid molecule comprising:
(i) a nucleic acid sequence represented by SEQ ID NO: 6, or the complement strand thereof;
(ii) a nucleic acid sequence encoding an amino acid sequence represented by SEQ ID NO: 7, or homologues, derivatives or active fragments thereof;
(iii) a nucleic acid sequence capable of hybridising (preferably under stringent conditions) with a nucleic acid sequence of (i) or (ii) above, which hybridising sequence preferably encodes a protein having GRUBX activity;
(iv) a nucleic acid sequence according to (i) to (iii) above which is degenerate as a result of the genetic code;
(v) a nucleic acid which is an allelic variant of the nucleic acid sequences according to (i) to (iv);
(vi) a nucleic acid which is an alternative splice variant of the nucleic acid sequences according to (i) to (v);
(vii) a nucleic acid sequence which has 75.00%, 80.00%, 85.00%, 90.00%, 95.00%, 96.00%, 97.00%, 98.00% or 99.00% sequence identity to any one or more of the sequence defined in (i) to (vi);
(viii) a portion of a nucleic acid sequence according to any of (i) to (vii) above, which portion preferably encodes a protein having GRUBX activity.

20. An isolated protein comprising at least part of one of the polypeptides selected from the group consisting of:
(i) a polypeptide as given in SEQ ID NO 4;
(ii) a polypeptide as given in SEQ ID NO 7;
(iii) a polypeptide with an amino acid sequence which has at least 40.00% sequence identity, preferably 50.00%, 60.00%, 70.00% sequence identity, more preferably 80% or 90% sequence identity, most preferably 95.00%, 96.00%, 97.00%, 98.00% or 99.00% sequence identity to the amino acid sequence as given in SEQ ID NO 4 or SEQ ID NO 7;
(iv) a polypeptide comprising at least an UBX domain, preferably an UBX domain and a PUG domain, and optionally a Zinc finger domain;
(v) a homologue, a derivative, an immunologically active and/or functional fragment of a protein as defined in any of (i) to (iv),
with the proviso that the protein sequence is not a sequence represented by SEQ ID NO 2, or database entries Q9ZU93, AAR01744, Q9D7L9, Q9BZV1, Q99PL6, ENSANGP00000020442, Q7SXA8, Q9V8K8, Q96IK9, ENSRNOP00000037228, or AAH07414.

21. A construct comprising:
(i) a nucleic acid molecule encoding a GRUBX protein;
(ii) one or more control sequences capable of driving expression in a plant of the nucleic acid molecule of (i); and optionally,
(iii) a transcription termination sequence,
provided that said nucleic acid encoding a GRUBX protein is not the nucleic acid molecule represented in GenBank Accession number AX927140.

22. A construct according to claim 21, wherein said nucleic acid molecule encoding a GRUBX protein encodes a protein represented by SEQ ID NO 2 or a protein according to any of (i) to (v) in claim 20.

23. A construct according to claims 21 or 22, wherein said control sequences comprise at least a seed-preferred promoter, preferably a prolamin promoter.

24. A construct comprising an expression cassette essentially similar to SEQ ID NO 5.

25. A transgenic plant or plant cell, characterized in that said plant or plant cell has increased expression of a nucleic acid sequence encoding a GRUBX protein and/or increased activity and/or levels of a GRUBX protein.

26. A transgenic plant or plant cell of claim 25 having improved growth characteristics.

27. A transgenic plant according to claim 25 or 26, wherein said plant is a crop plant comprising soybean, sunflower, canola, alfalfa, rapeseed or cotton, preferably a monocotyledonous plant such as sugarcane, most preferably a cereal, such as rice, maize, wheat, millet, barley, rye, sorghum or oats.

28. Plant cells, plant parts, including harvestable parts and/or products directly derived there from, propagules or progeny of a plant according to claim 25 or 26.

29-34. (canceled)


SEQUENCE LISTING (7 sequences)

SEQ ID NO: 1 - DNA, 1380 bp, Nicotiana tabacum
atgggtgacatgaaggataaagtcaaagggttcatgaaaaaagtcacatcttcttcttcaggtaagtttaaaggccaaggtagggttttgggtggttcatcttcttcaggaccctcaaatcatgtcaataatttttcatcacatcccctaaatacaaggcaagatcaacaaccttcatatacaaaaacttcgcctcaaaaaccaagtaattctgatcaaagaattgagaatatatgtgaaattcagttcaacaaaagtgaatcaaaggatggttttgatccatttggtgaattagtcacttctgggaagagaaacccaaaagggtattcacttactaatgtgtttgaatgccctgtctgtggtagtggttttgtttctgaagaagaggtgtcaactcatattgatagctgtttaagttctgaagtgtcttctaatttgggagttgaaagtaaagttgaagttaaaagtgaattggaaacatgtgttagtgcatatgtttcagggaagccctcagaagggtcagttgaagtggtcattaagttgttaaagaatattgtgaaggaaccagagaatgccaagtttaggaaaataaggatggggaatccaaaaataaaaggtgctataggtgatgttgtaggaggagtggagctattggaatttgttggatttgagttgaaagaagaaggtggggaaatttgggctgtgatggatgttccttctgaagaacaacttgttatgcttaagaatgtagtttcactcttggaaccgaagaaggttgaagagttggcgtccttatcccaagttaaggcgagtgaaccagttgagccgaagaagattgatagacagattcgagtgttcttttctgttcccgagagcgtagcagcaaaaattgagctacctgattccttctttaacctctcacgtgaggaattgagaagagaagcagagatgaggaagaagaaattagaagattccaaattattgattcctaaatcttatcgggaaaagcaggcaaaagctgcaagaaagaagtacacaaaatccattatccgtgtacagtttccagatggagcattgcttcaaggtgtctttctaccttcggagccaactagtgctctttatgagtttgtgagcgcagcgttaaaggaaccaagcttagagttcgaattgttacatccggtgcttgttaaaaagcgggtgattccccattttccagctgctggggagagggctgtaacagttgaagaggaggatttggttcctgcagctctactcaaatttaaacctatcgaaacagattctgttgtttttactggtctttgtaatgagcttcttgaaattagcgagcccctcgagaccggatcagttgcttcctcgtaa

SEQ ID NO: 2 - protein, 459 aa, Nicotiana tabacum
MGDMKDKVKGFMKKVTSSSSGKFKGQGRVLGGSSSSGPSNHVNNFSSHPLNTRQDQQPSYTKTSPQKPSNSDQRIENICEIQFNKSESKDGFDPFGELVTSGKRNPKGYSLTNVFECPVCGSGFVSEEEVSTHIDSCLSSEVSSNLGVESKVEVKSELETCVSAYVSGKPSEGSVEVVIKLLKNIVKEPENAKFRKIRMGNPKIKGAIGDVVGGVELLEFVGFELKEEGGEIWAVMDVPSEEQLVMLKNVVSLLEPKKVEELASLSQVKASEPVEPKKIDRQIRVFFSVPESVAAKIELPDSFFNLSREELRREAEMRKKKLEDSKLLIPKSYREKQAKAARKKYTKSIIRVQFPDGALLQGVFLPSEPTSALYEFVSAALKEPSLEFELLHPVLVKKRVIPHFPAAGERAVTVEEEDLVPAALLKFKPIETDSVVFTGLCNELLEISEPLETGSVASS

SEQ ID NO: 3 - DNA, 1311 bp, Saccharum officinarum
  misc_feature (277)..(279): n can be any nucleotide
atgatgaaggacaagatgaaggagttcatgaagaaggtcacctcctccgggtccgggaccccctcctccttcaagggcacctcccacgtcctcggctccggcccctccccctcctcctcccaccccgctgcccgctcctcaaaccctagcccaaacctcaggcccgctcctaagcggacctcgccacctaccccgcccactttaaccaccgatttgacctccttcacgcccctcgtctgctactcctcccgccgccccgacgcgaacggcaccgcgnnngccgtcgccaccgtcgcgtgccccagctgcggagacgcgtttccgtccgagctcgccgtctccgagcatctcgacggctgcctcgcgtcggcggggggcgcccgcgcgcgcgccgccgcgtacctcgccgccgacccgcctccgcccgcggcctccgtagaggtggtcaaacgcctgctgggcaacctgctccgggagcccggcaacgataagttcaggcgggtgagattgggtaacccgcggatcaaggaggccctggcagacagggatggcggggtggagctcctggaggccgtcggcttcacagttggggatgagggcggggagcccttcgccgtgatggacgaagtgcctagcgaccctaggctcaacgggatcaggagggccgtcctcctgctcgagggggcacacccctctgcgcctccagtgaaggcggaggctgaggccaaggagagctgcagcaatgtgtctgacgtgcaggagggtgctaagactattgatcggcagattcgggtatttgtctctgttcctgggagttctatggcacaaaatgatgtaccagattctttttacaagcttagtggtgaggagataaggaatgaagcaaagatgaggagggaaaggttagaacaatctcgattgctgataccaaagtcttacaaggagaaacaggcattggctgctcgacagaagtataaacaagcagtcattcgagttcagtttccagatagaatgattcttcagggcatattcctaccaggagaggccactagttcactgtatgagttcgtcacatctgctctgaagcaatcaggtttggaattcgaacttatctctccagccatacctaagccacgtgtggtgccccattttccaaacccgggagagcgggcacgcaccttgcaagaggaggagctggtcccatctgcgctcctcaagttcattcccaaggagactgattccatggttttcaccggtttgcttgatgagcttctcatggccagtgagccgcttcctgctgcatcacaatga

SEQ ID NO: 4 - protein, 436 aa, Saccharum officinarum
  MISC_FEATURE (93)..(93): Xaa can be any amino acid
MMKDKMKEFMKKVTSSGSGTPSSFKGTSHVLGSGPSPSSSHPAARSSNPSPNLRPAPKRTSPPTPPTLTTDLTSFTPLVCYSSRRPDANGTAXAVATVACPSCGDAFPSELAVSEHLDGCLASAGGARARAAAYLAADPPPPAASVEVVKRLLGNLLREPGNDKFRRVRLGNPRIKEALADRDGGVELLEAVGFTVGDEGGEPFAVMDEVPSDPRLNGIRRAVLLLEGAHPSAPPVKAEAEAKESCSNVSDVQEGAKTIDRQIRVFVSVPGSSMAQNDVPDSFYKLSGEEIRNEAKMRRERLEQSRLLIPKSYKEKQALAARQKYKQAVIRVQFPDRMILQGIFLPGEATSSLYEFVTSALKQSGLEFELISPAIPKPRVVPHFPNPGERARTLQEEELVPSALLKFIPKETDSMVFTGLLDELLMASEPLPAASQ

SEQ ID NO: 5 - DNA, 3048 bp, Artificial sequence
  expression cassette comprising GRUBX (1011-2390) operably linked to the prolamine promoter (1-654) and the T-Zein + T-Rubisco deltaG terminator (2615-2808 and 2852-3048)
cttctacatcggcttaggtgtagcaacacgactttattattattattattattattattattattttacaaaaatataaaatagatcagtccctcaccacaagtagagcaagttggtgagttattgtaaagttctacaaagctaatttaaaagttattgcattaacttatttcatattacaaacaagagtgtcaatggaacaatgaaaaccatatgacatactataattttgtttttattattgaaattatataattcaaagagaataaatccacatagccgtaaagttctacatgtggtgcattaccaaaatatatatagcttacaaaacatgacaagcttagtttgaaaaattgcaatccttatcacattgacacataaagtgagtgatgagtcataatattattttctttgctacccatcatgtatatatgatagccacaaagttactttgatgatgatatcaaagaacatttttaggtgcacctaacagaatatccaaataatatgactcacttagatcataatagagcatcaagtaaaactaacactctaaagcaaccgatgggaaagcatctataaatagacaagcacaatgaaaatcctcatcatccttcaccacaattcaaatattatagttgaagcatagtagtaatttaaatcaactagggatatcacaagtttgtacaaaaaagcaggctggtaccggtccggaattcccgggatatcgtcgacccacgcgtccgccaatatcagatttctttcatgaactccacttccaatttctcattgcttcttcttcccatttccacctccaaagccatccttccagaaaaccttgttccttacatttcttagccccaaaaaagattcccatctcaattccacaaaaaaacacaaggagatctaaggaaattccccgcctctatatatagagaggtggaattgttcctgaatttggtttgaattgattgattgacagattttggtgagagggtgttattgaaaaaatgggtgacatgaaggataaagtcaaagggttcatgaaaaaagtcacatcttcttcttcaggtaagtttaaaggccaaggtagggttttgggtggttcatcttcttcaggaccctcaaatcatgtcaataatttttcatcacatcccctaaatacaaggcaagatcaacaaccttcatatacaaaaacttcgcctcaaaaaccaagtaattctgatcaaagaattgagaatatatgtgaaattcagttcaacaaaagtgaatcaaaggatggttttgatccatttggtgaattagtcacttctgggaagagaaacccaaaagggtattcacttactaatgtgtttgaatgccctgtctgtggtagtggttttgtttctgaagaagaggtgtcaactcatattgatagctgtttaagttctgaagtgtcttctaatttgggagttgaaagtaaagttgaagttaaaagtgaattggaaacatgtgttagtgcatatgtttcagggaagccctcagaagggtcagttgaagtggtcattaagttgttaaagaatattgtgaaggaaccagagaatgccaagtttaggaaaataaggatggggaatccaaaaataaaaggtgctataggtgatgttgtaggaggagtggagctattggaatttgttggatttgagttgaaagaagaaggtggggaaatttgggctgtgatggatgttccttctgaagaacaacttgttatgcttaagaatgtagtttcactcttggaaccgaagaaggttgaagagttggcgtccttatcccaagttaaggcgagtgaaccagttgagccgaagaagattgatagacagattcgagtgttcttttctgttcccgagagcgtagcagcaaaaattgagctacctgattccttctttaacctctcacgtgaggaattgagaagagaagcagagatgaggaagaagaaattagaagattccaaattattgattcctaaatcttatcgggaaaagcaggcaaaagctgcaagaaagaagtacacaaaatccattatccgtgtacagtttccagatggagcattgcttcaaggtgtctttctaccttcggagccaactagtgctctttatgagtttgtgagcgcagcgttaaaggaaccaagcttagagttcgaattgttacatccggtgcttgttaaaaagcgggtgattccccattttccagctgctggggagagggctgtaacagttgaagaggaggatttggttcctgcagctctactcaaatttaaacctatcgaaacagattctgttgtttttactggtctttgtaatgagcttcttgaaattagcgagcccctcgagaccggatcagttgcttcctcgtaagctctaaattacatcagactttgaattcttctgagtgttggaaaccttataaaactctctgcgccgggaatgctgcggccgctctagagtatccctcgaggggcccaagcttacgcgtacccagctttcttgtacaaagtggtgatatcacaagcccgggcggtcttctagggataacagggtaattatatccctctagatcacaagcccgggcggtcttctacgatgattgagtaataatgtgtcacgcatcaccatgggtggcagtgtcagtgtgagcaatgacctgaatgaacaattgaaatgaaaagaaaaaaagtactccatctgttccaaattaaaattcattttaaccttttaataggtttatacaataattgatatatgttttctgtatatgtctaatttgttatcatccgggcggtcttctagggataacagggtaattatatccctctagacaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatactcgaac

SEQ ID NO: 6 - DNA, 1302 bp, Oryza sativa
atgatgaaggaaaagatgaaggatctcatgaggaaggtcacctcctcctcctcctcctcctcgtcgtcctcctccttcaagggcaccgcccacgtcctcggctccggccccgacccctcctcccgcccctccaaccctacccctagtcgccccgctgccccccggcgagaggccgccgcctccgcgaggccgccctcctccggcttcgccccctactccccgctcatctccacctcctcccgccgcaccgacccacccgcgggggcgggggcgggggaggacgacgccgtcgcgtgccccagctgcgccgagccgttcccctccgagctggcggtgtcggaccacctcgacggctgcctcgcggcggcggggggagcccgcccccgcgcggccgcctacctggccggcgacccccccgcgtccgccgtggaggtggtgaagaggctgctcgggaacctgctctccgacccccggaacgacaagtacaggaaggtcaggctcgggaacccgaggatcaaggaggccctggcggacagggagggcggggtggatctcctcgaggccgtggggttcagggtcgccgacgagggcggggagctcttcgccctcatggacgaggtgcccggggacgcgaggctcggcggcatcaggcaggccgtgctcctgctcgagagggcccggccatcgacgccgccgcagacacaggcagatgccaaagagacttgcccgaatggagttagcgaagagcaggggattaagaagccggttgatcgtcagattcgggtgttcttctctgttgctgcaagttctgttgcagaaaatgatctaccagattctttctatagccttagtaatgaggagatcaggaatgaggcaaagatgaggagggagaggctagaacaatctcggttgttgattccaaagtcatataaggagaagcaggcactggctgcccgacagaagtataaacaagctctgattcgaattcagtttccggatggagtaattctgcagggtgtgttccttcccgcggagcccattagttcactatatgagtttgtcgcatcttctttgaagcagccaagtttggaattcgatcttatctgtccagcgggtcctaggacacgcgtcatacctccttttccaaaaccaggggaacaggcacgcacactgcgggatgaagacctagtcccttctgctcggctcaccttcaagcccaaagagactgattcggttgtgttcactggtctgttggacgagcttcttgagaccagtgagccgttcacatctgcatcctcatga

SEQ ID NO: 7 - protein, 433 aa, Oryza sativa
MMKEKMKDLMRKVTSSSSSSSSSSSFKGTAHVLGSGPDPSSRPSNPTPSRPAAPRREAAASARPPSSGFAPYSPLISTSSRRTDPPAGAGAGEDDAVACPSCAEPFPSELAVSDHLDGCLAAAGGARPRAAAYLAGDPPASAVEVVKRLLGNLLSDPRNDKYRKVRLGNPRIKEALADREGGVDLLEAVGFRVADEGGELFALMDEVPGDARLGGIRQAVLLLERARPSTPPQTQADAKETCPNGVSEEQGIKKPVDRQIRVFFSVAASSVAENDLPDSFYSLSNEEIRNEAKMRRERLEQSRLLIPKSYKEKQALAARQKYKQALIRIQFPDGVILQGVFLPAEPISSLYEFVASSLKQPSLEFDLICPAGPRTRVIPPFPKPGEQARTLRDEDLVPSARLTFKPKETDSVVFTGLLDELLETSEPFTSASS